(19) United States
(12) Patent Application Publication
(10) Pub. No.: US 2022/0249352 A1
(43) Pub. Date: Aug. 11, 2022

(54) MICROPARTICLES BASED ON ESTER DERIVATIVES OF HYALURONAN, METHOD OF PRODUCTION, COMPOSITION COMPRISING THEREOF AND USE THEREOF

(71) Applicant: CONTIPRO A.S., Dolni Dobrouc (CZ)

(72) Inventors: Gloria HUERTA ANGELES, Ceska Trebova (CZ); Martina BRANDEJSOVA, Letohrad (CZ); Paulina ORZOL, Gliwice (PL); Katerina KOPECKA, Nemcice (CZ); Vojtech PAVLIK, Usti nad Orlici (CZ); Jaroslav NOVOTNY, Letohrad (CZ); Iva DOLECKOVA, Policka (CZ); Kristina NESPOROVA, Usti nad Orlici (CZ); Vladimir VELEBNY, Zamberk (CZ)

(73) Assignee: CONTIPRO A.S., Dolni Dobrouc (CZ)

(21) Appl. No.: 17/439,284

(22) PCT Filed: Mar. 13, 2020
(86) PCT No.: PCT/CZ2020/050010

§ 371 Date: Sep. 14, 2021

(30) Foreign Application Priority Data
Mar. 14, 2019 (CZ) PV2019-153

(51) Int. Cl. A61K 8/73; A61K 8/36; A61Q 19/08; A61Q 17/00

(52) U.S. Cl. CPC A61K 8/735; A61K 8/361; A61K 2800/654; A61Q 17/005; A61Q 19/08

(57) ABSTRACT

Microparticles based on ester derivatives of hyaluronan and related methods and compositions are disclosed. The microparticles comprise a conjugate of all-trans retinoic acid and a particular hyaluronan comprising from 1 to 5000 dimer units; where the microparticles comprise a degree of substitution of all-trans retinoic acid residues in the conjugate of hyaluronan in the range of from 0.1 to 8%. Methods of preparing the microparticles and related compositions are also disclosed.

### CROSS-REFERENCE TO RELATED APPLICATIONS

This application is the National Stage of International Application No. PCT/CZ2020/050010, filed on 13 Mar. 2020, which claims priority to and all advantages of CZ Application No. PV2019-153, filed on 14 Mar. 2019, the contents of which are hereby incorporated by reference.

### TECHNICAL FIELD

The present disclosure relates to microparticles based on esters of hyaluronan, a method of its production, composition comprising thereof and use thereof. Particularly, the microparticles containing all-trans retinoic acid and covalently joint to hyaluronan, thus a conjugate of all-trans retinoic and hyaluronan (the conjugate of HA-ATRA or HA-ATRA).

### BACKGROUND

Hyaluronan is a linear polysaccharide that is present in all living subjects was chemically modified in one step. Hyaluronan is present in the synovial fluid, which lubricates and cushions joints. However, hyaluronan easily degrades and native HA is not characterized to have any antioxidant properties by itself. It is also desirable that hyaluronan could carry and deliver therapeutic agents useful in the treatment of several medical and cosmetic applications.

All-trans retinoic acid (ATRA, tretinoin), a derivative of vitamin A, is a common component in cosmetics and commercial acne creams as well as a first-line chemotherapeutic agent or for conditions of the respiratory tract (WO2003037385A1, US20030161791A1) or in compositions used to treat ocular disorders (US20140330005A1). The administration of ATRA presents many difficulties due to its hydrophobic nature and poor stability. Nowadays, formulations made for the topical application of ATRA are based on creams and emulsions applied directly to deliver the compound to/into the skin. Thus, ATRA is immediately taken up. Unfortunately, many adverse side-effects of ATRA have been observed such as skin irritation, hair loss and desquamation. Thus, the current research has been focused on EGFR tyrosine kinase inhibitors to mitigate the above-mentioned adverse side effects (WO2009091889, US2009/031101). Besides, the patent document US2019/0015366A1 provides an encapsulated tretinoin composition, said composition comprising microcapsules comprising a core comprising tretinoin coated by a shell, wherein said core is in a solid form and said microcapsules have a size of less than about 50 μm. Even though several works have demonstrated the feasibility to prepare tretinoin-loaded nanocapsules, the encapsulation of the active compound is still considered low.

In this case, several agents have been reported and they comprise, as a main ingredient i.e. a polyvalent metal inorganic-salt nanocapsule which encapsulates a retinoid such as retinoic acid for cartilage injection (US20110081410A1).

In similar art, the combination of retinoic acid (RA) with low molecular weight compounds such as hydroquinone (HQ), forming a codrug (an ester) was studied or combined with carnitine and acyl carnitines (EP963754A1). Similarly, synthetic low molecular weight analogous such as esters or amides of ATRA have been prepared (U.S. Pat. Nos. 4,108,880 and 4,055,659). Both patents, are related to topical applications of retinoids for treatment of acne and skin diseases, more specifically, this patent described esters of 13-trans-retinoic acid. For example, tretinoin is used in prescription acne products as well as prescription anti-wrinkle products and is used to fade the look of wrinkles in skin, smooth fine lines, improve skin texture, and brighten skin tone. However, the use of gluconolactone or glucarolactone in cosmetic skin care compositions, as anti-irritants, have been used to reduce skin irritation, which may be intrinsic skin irritation or irritation caused by hydroxy acids or certain retinoids (U.S. Pat. No. 6,036,963A).

The patent applications KR20180111584 and US20180280276A1 reported the use of super-hydrophilic polymers comprised of repeat units comprising multiple hydroxyl functionalities, for example, starch, hydroxyethylcellulose, dextran, inulin, pullulan, poly(glyceryl methacrylate), poly[tris(hydroxymethyl)acrylamidomethane)], or poly(sucrose methacrylate), with reagents that will result in amphiphilic repeat units. However, in some case the activity of retinoids remains low (US20180280275A1) or polyamines or polymers containing amines (U.S. Pat. No. 6,344,206B1).

Still, unsatisfactory outcomes and safety concerns have been reported due to the instability of tretinoin. In similar art, U.S. Pat. No. 3,729,568 discloses the use of retinoic acid derivatives i.e. the use of 4-nitrobenzyl all-trans-retinoate for the treatment of acne. Even though, ATRA is also known to have ultraviolet (UV) absorption properties, it is not useful as a sunscreen agent because of its irritating effects and fast degradation when exposed to sun light.

Polysaccharidic esters of retinoic acid were reported in U.S. Pat. No. 6,897,203. Specifically, ester, and amide derivatives of hyaluronan were described. Several inconveniencies can be found in this application HA is converted to its tetrabutylammonium salt towards its dissolution in N, N-dimethylformamide to make it soluble in highly polar organic solvents particularly in N, N-dimethylformamide. Dimethyl formamide is a solvent that produces hepatotoxicity and many toxic reactions in humans and animals. Additionally, the esterification reaction was carried out by reaction of the alcoholate with retinoyl chloride. Retinoyl chloride is formed by activation of retinoic acid with oxalyl chloride. Oxalyl chloride produce acute bronchiolitis when the chemical compound was tested in animals. Then, it is a matter of concern to have residues of this chemical. As pulmonary edema appears to contribute significantly to mortality caused by oxalyl chloride. Furthermore, the formation of the retinoyl chloride may be performed by using chlorinating agents i.e. by the action of dimethylchloroformamidinium chloride (III). As previously reported in the patent no. EP0261911B, dimethylchloroformamidinium chloride (III) is extremely hygroscopic and those facts considerably complicates the handling of the compound. Moreover, N,N′,N′-tetramethylformamidinium chloride, a very toxic compounds is also obtained by the reaction of dimethylformamide (DMF) with dimethylcarbamoyl chloride.

In similar art, U.S. Pat. No. 6,897,203B2 described the substitution of the hydroxyl groups in HA by a selective halogenation reaction which is performed by the following steps: suspension of the polysaccharide in organic solvent under stirring for 1-5 hours at 25-100° C., addition of a halogenating agent at a temperature that can vary from −20° C. to 100° C. under constant stirring for 1-20 hours and possible alkalynisation of the reaction mixture at a pH ranging from 9 to 11, which may induce degradation of the polysaccharide. At the end, the reaction mixture is neutralized, and the activated polysaccharide is recovered according to conventional procedures. As people skilled in that art knows in this reaction halogenating agents such as ethanesulphonyl bromide, methanesulphonyl chloride, p-toluenesulphonyl bromide, p-toluenesulphonyl chloride, thionyl chloride, thionyl bromide are required. Unfortunately, they are extremely toxic. Furthermore, these agents are moisture sensitive, corrosive, and lachrymator reagents. On the other hand, the process of purification reported in the manuscript published in Ventura C, Maioli M, Asara Y, Santoni D, Scarlata I, Cantoni S, et al. Butyric and retinoic mixed ester of hyaluronan. A novel differentiating glycoconjugate affording a high throughput of cardiogenesis in embryonic stem cells. J Biol Chem 2004; 279:23574-9, does not warranty the required pharmaceutical purity of the final product. In other words, if the polymer is only precipitated into three volumes of diethyl ether or acetone and recuperated by suction filtration. The product will retain the DMF used in the reaction as well as the base. Additionally, a process of scale up by precipitation of a product with diethyl ether is not possible due to the explosivity of the solvent.

U.S. Pat. No. 6,897,203 describes the induction of cardiac differentiation of embryonal pluripotent murine teratocarcinoma cells by the presence of polysaccharidic esters. However, embryonal pluripotent murine teratocarcinoma cells cannot be considered as an in vitro model for skin application. (Development of an in vitro model for studying the penetration of chemicals through compromised skin, Toxicology in Vitro Volume 29, Issue 1, February 2015, Pages 176-181, Design of in vitro skin permeation studies according to the EMA guideline on quality of transdermal patches, European Journal of Pharmaceutical Sciences Volume 125, 1 Dec. 2018, Pages 86-92).

U.S. Ser. No. 14/106,064A, US20100298249A1 refer to pharmaceutical/cosmetic compositions containing a dermatologically effective amount of hyaluronic acid, at least one retinoid and/or salt and/or derivative thereof, at least one oligosaccharide and at least one inhibitor of hyaluronic acid degradation, formulated into a physiologically acceptable medium therefor, are useful for the treatment of wrinkles, fine lines, fibroblast depletions and scars. However, this formulation includes an inhibitor of HA degradation. The inventive compositions for topical application are characterized in that they comprise one or several hyaluronate fragments in the form of a main principle whose molecular weight ranges from 50000 and 750000 Da and a retinoid if necessary.

U.S. Pat. No. 8,968,751B2 describes several pharmaceutical/cosmetic compositions containing a dermatologically effective amount of hyaluronic acid, at least one retinoid and/or salt and/or derivative thereof, at least one oligosaccharide and at least one inhibitor of hyaluronic acid degradation, formulated into a physiologically acceptable medium therefor, are useful for the treatment of wrinkles, fine lines, fibroblast depletions and scars. However, they include the use of the unstable retinaldehyde. Some other patent documents only include the use of native hyaluronan (U.S. Pat. No. 6,680,062B2).

Additionally, WO2005092283A1 is directed to compositions which contain a combination of at least one histone deacetylase inhibitor (HDAC inhibitor) and a retinoid. Particularly, the composition is a cosmetic preparation. In this case, an additional amount of antioxidants/preservatives is generally preferred, which may be present in an amount about 0.01 wt. % to about 10 wt. % of the total weight of the composition of the disclosed invention. Preferably, one or more preservatives/antioxidants are present in an amount about 0.1 wt. % to about 1 wt. %. The same was reported in the patent KR19990087346A, wherein the stability of retinoids is increased by the incorporation of hydroxy toluene (Butylated Hydroxy-toluene; BHT) or the use of histidine (U.S. Pat. No. 6,358,514B1).

Moreover, amphiphilic polymer coating of coated vitamin A micelle can be used for containing A retinoid and increase its stability (CN103565676A). However, the biological activity and compatibility of the amphiphilic polymer coating was not reported. As people skilled in the art is aware, cream formulations containing—tretinoin possess some undesirable attributes. As an example, cream formulations of tretinoin are limited due to their relative instability, often necessitating the use of refrigeration or antimicrobial preservatives to prevent microbiological contamination, as well as special additives to maintain physical stability. One way of overcoming some or all these undesirable attributes is i.e. by using gel formulations (U.S. Pat. No. 4,073,291).

### BRIEF SUMMARY

The problems mentioned above are solved in the present embodiments concerning microparticles based on ester derivatives of hyaluronan or its salt. Specifically, a composition comprising microparticles based on ester derivatives of hyaluronan is provided. The microparticles comprise a conjugate of all-trans retinoic acid and hyaluronan of the general formula I:

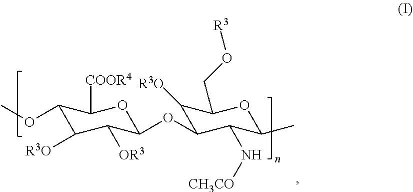

wherein n is integer in the range of from 1 to 5000 dimers,
each R⁴ is H⁺ or a pharmaceutically acceptable salt,
each R³ is —H or an all-trans retinoic acid residue of the formula II, where is in the place of covalent bond of all-trans retinoic acid residue of the formula II

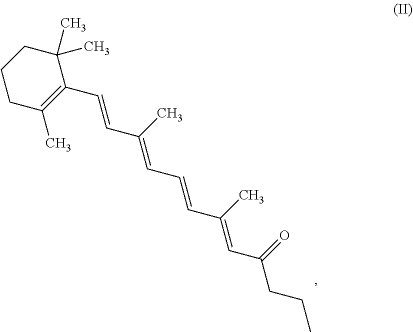 [SMILES: CCCC(=O)/C=C(C)/C=C/C=C(C)/C=C/C1=C(C)CCCC1(C)C]

with the proviso that at least one R³ of the conjugate is the all-trans retinoic acid residue of the formula II, and wherein the degree of substitution of the all-trans retinoic acid residues of the formula II in the conjugate of hyaluronan is in the range of from 0.1 to 8%.

A method of preparing the composition, and particular forms of the composition for cosmetic and/or therapeutic use are also provided.

### BRIEF DESCRIPTION OF THE DRAWINGS

FIG. 1 provides ¹H NMR of HA-ATRA microparticles.

FIG. 2 provides ¹H NMR of HA-ATRA granules and microparticles after 12 months of preparation (storage at 25° C.).

FIG. 3 provides an analysis of UV of HA-ATRA for the structural determination of total concentration of ATRA-HA microparticles and stability.

FIG. 4 provides a TGA analysis of HA-ATRA (granules) and HA-ATRA microparticles.

FIG. 5 provides SEM images of spray-dried microparticles in the form of powders (DS=0.5%).

FIG. 6 provides SEM images of spray-dried microparticles in the form of powders (DS=2.0%).

FIG. 7 provides SEM images of spray-dried microparticles in the form of powders (DS=6.1%).

FIG. 8 shows an effect of Mw on the stability of the microparticles.

FIG. 9 provides a determination of biocompatibility in NIH-3T3 cells for the derivatives (A) HA-ATRA of Examples 5 and 9 and ATRA dissolved in DMSO, which was used as control.

FIG. 10 shows the gene expression of luciferase reporter under RARE element described in Example 14. ATRA, HA−ATRA or unconjugated HA+ATRA were incubated in decreasing concentrations. HA−ATRA can induce gene expression in dose-dependent fashion.

FIG. 11 shows the expression of genes HMGCS1 and SQLE involved in cholesterol synthesis after cell treatment with the microparticles described in Example 15. Only HA−ATRA derivative could increase gene expression of the cholesterol metabolism genes. All treatments with retinoic acid or its isomers induced expression of DHRS3, involved in retinoid metabolism, which proves sensitivity of the experimental system to detect gene expression changes.

FIG. 12 shows expression of HMGCS1 in fibroblasts after treatment with the microparticles described in Example 15 with varying DS. Concentration corresponds to micromoles of added retinoic acid. Effect on HMGCS1 expression can be reached by derivate of DS 0.45% and DS 6.8%.

FIG. 13 provides skin penetration of Nile red—loaded in HA−ATRA to the dermis.

FIG. 14 shows NIH-3T3 fibroblasts treated under UV and hydrogen peroxide, generated less reactive oxygen species (ROS) after incubation with HA−ATRA (DS=0.5%).

FIG. 15 shows DPPH assay results showing antioxidant activity of HA−ATRA, (DS=0.5%) FIG. 16 shows expression of collagen 1 after incubation with HA−ATRA, DS=0.5%.

FIG. 17 shows expression of elastin after incubation with HA−ATRA, DS=0.5%.

FIG. 18 shows expression of fibronectin after incubation with HA−ATRA, DS=0.5%.

FIG. 19 shows expression of IL-8 after treatment with HA−ATRA, DS=0.5%.

FIG. 20 shows the antimicrobial effect observed for HA−ATRA, DS=2.0% in respect to control.

FIG. 21 provides a dermal irritation test of HA−ATRA microparticles.

FIG. 22 provides a determination of Mw of microparticles of Examples 1 at time 0; Mw=15,350 g/mol and polydispersity=Mw/Mn=1.595 and after 3 months at 40° C.; Mw=16,660 g/mol and polydispersity=Mw/Mn=2.178.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The subject-matter of the embodiments concerns microparticles comprising the conjugate of all-trans retinoic acid and hyaluronan of the general formula I:

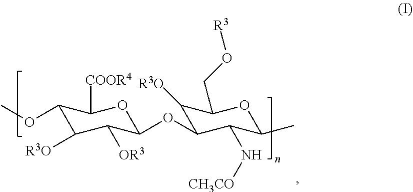

wherein n is integer in the range of 1 to 5000 dimers,
R⁴ is H⁺ or a pharmaceutically acceptable salt,
R³ is —H or all-trans retinoic acid residue of the formula II, where is in the place of covalent bond of all-trans retinoic acid residue of the formula II

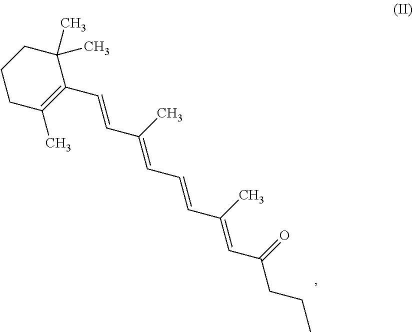 [SMILES: CCCC(=O)/C=C(C)/C=C/C=C(C)/C=C/C1=C(C)CCCC1(C)C]

providing that at least one R³ of the conjugate is all-trans retinoic acid residue of the formula II and wherein the degree of substitution of all-trans retinoic acid residue of the formula II in the conjugate of hyaluronan is in the range from 0.1 to 8%.

A molar weight of the conjugate of the general formula I is in the range of 3,200 g/mol to 100,000 g/mol, preferably in the range from 6,000 to 20,000 g/mol, more preferably 15,000 g/mol.

The degree of substitution in the conjugate of hyaluronan of the general formula I is in the range from 0.5 to 8% preferably the degree of substitution is in the range of 0.5 to 6.5%, when the molar weight of the conjugate of the general formula I is in the range of 6,000 g/mol to 30,000 g/mol, preferably 6,000 g/mol to 20,000 g/mol.

Preferably the degree of substitution in the conjugate of hyaluronan of the general formula I is in the range from 0.3% to 3.1%, preferably 0.3% to 2.5% when the molar weight of the conjugate of the general formula I is in the range of 6,000 g/mol to 30,000 g/mol of 6,000 g/mol to 20,000 g/mol.

The pharmaceutically acceptable salt of the conjugate of the general formula I is selected from a group comprising any of ions of alkali metals or ions of alkaline-earth metals, preferably Na⁺, K⁺, Mg²⁺ or L⁺.

The average diameter of the microparticles according to the present embodiments is in the range of 500 nm to 5 μm, preferably 800 nm to 2 μm.

The microparticles according to the present embodiments contains 85 to 90 wt. % of dry matter, preferably the conjugate of the general formula I (HA−ATRA). And the rest is water.

In the literature, it is more often found the amount of retinoyl because it is considered as the active compound. Therefore, the amount retinoyl (ATRA) in microparticles is also expressed in in the Examples. The microparticles according to the present embodiments contain 0.5 to 10 wt. % of retinoyl, preferably 0.5 to 7 wt. %.

The microparticles according to the present embodiments can be used in several biological and medical applications. Preferably the microparticles or the compositions according to the present embodiments can be used for treatment of skin diseases or skin disorders selected from a group comprising hyperproliferative skin disorders, preferably psoriasis or skin inflammatory disorders preferably acne, post-inflammatory hyperpigmentation, dermatoheliosis (photoaging), melasma.

Another aspect if the present disclosure is a method of production of the microparticles according to the present disclosure comprising a reaction of an activated all-trans retinoic acid of the general formula III

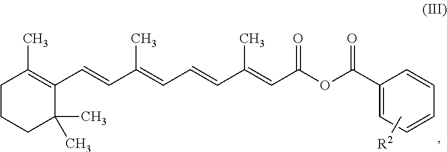

wherein R² is one or more substituents selected from a group comprising H, —NO₂, —COOH, halides, C₁-C₆alkylkoxy, preferably halides, methoxy or ethoxy, more preferably Cl;
with hyaluronic acid or the pharmaceutically acceptable salt thereof in the presence of an organic base in a mixture of water and water-miscible polar solvent in a ratio 99% to 50% v/v of water-miscible polar solvent, particularly 50% v/v to form a solution comprising the conjugate of all-trans retinoic acid and hyaluronan of the general formula I according to this disclosure; then spray-drying the solution at inlet temperature of 150° C. to 200° C., particularly 180° C. and outlet temperature of 80° C. to 100° C., particularly 90° C., forming of the microparticles of the conjugate of all-trans retinoic acid and hyaluronan of the general formula I.

In one aspect of the present embodiments, the lower limit of the molecular weight of the hyaluronan useful herein is from 6,000 g/mol, 10,000 g/mol, 20,000 g/mol, 50,000 g/mol, 60,000 g/mol, 70,000 Da, 80,000 g/mol, 90,000 g/mol, or 100,000 g/mol, and the upper limit is 200,000 g/mol, 300,000 g/mol, 400,000 g/mol, 500,000 g/mol, 600,000 g/mol, 700,000 g/mol, 800,000 g/mol, 900,000 g/mol, 1,000,000 g/mol, 2,000,000 g/mol where any of the lower limits can be combined with any of the upper limits. In one aspect, the hyaluronan has a molecular weight of 6,000 g/mol to 100,000 g/mol, more particularly, 15,000 g/mol.

The molecular weight of the hyaluronan used in the reaction with the activated all-trans retinoic acid of the general formula III as described above basically correspond to the molecular weight of the conjugate according to the present embodiments. In the course of time Mw of the conjugate can be slightly higher due to possible mutual cross-linking of the conjugate. For example starting with the conjugate of 15,000 g/mol after 6 months obtaining 17,000 to 21,000 g/mol.

The concentration of the conjugate of all-trans retinoic acid and hyaluronan is in the range of 0.25% to 2.5% (w/v), preferably 0.25 to 1.0 (w/v) in the solution after the reaction. The reaction of the activated all-trans retinoic acid of the general formula III and hyaluronic acid or the pharmaceutically acceptable salt thereof is carried out in the range of temperatures 0° C. to 37° C., preferably at 5° C. to 25° C., more preferably at 5° C. to 10° C., for 1 to 4 hours, in darkness.

The organic base is selected from the group comprising aliphatic amine having a linear or branched, saturated or unsaturated, C₃-C₃₀ alkyl group, preferably it is selected from the group comprising N,N-diisopropylethylamine, triethylamine, dimethylaminopyridine,

The polar solvent is preferably isopropanol. and, the polar solvent is selected from the group comprising isopropanol, dimethyl sulfoxide, tert-butanol, dioxane and tetrahydrofuran and it is preferably isopropanol

The molar amount of the activated all-trans retinoic acid of the general formula III is 0.01 to 2.0 equivalents, preferably 0.03 to 0.3 equivalents with respect to a dimer of hyaluronic acid.

The activated all-trans retinoic acid of the formula III is formed by activation reaction of all-trans retinoic acid with an activation agent, is a substituted or non-substituted benzoyl chloride or its derivatives of the general formula IV

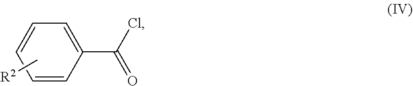

wherein R² is one or more substituents selected from a group comprising H, —NO₂, —COOH, halides, C₁-C₆alkylkoxy, preferably halides, methoxy or ethoxy, more preferably Cl, preferably benzoyl chloride, in the presence of an organic base and a mixture of water and water-miscible polar solvent.

The substituents R² of benzoyl chloride or its derivatives of the general formula IV as defined above can be located in positions ortho-, metha- or para- to the acyl chloride-group, preferably in ortho- or para-positions. The use of benzoyl chloride and its derivatives as the activators is not generally used for chemical modification of HA because it is believed that catalyzes transesterification reactions and it may react with common organic solvents used for the chemical modification of HA and respective isolation and purification, such as ethanol, methanol and higher alkyl-alcohols.

The forming of the activated all-trans retinoic acid of the general formula III, as defined above, is carried out at the temperature in the range of 5° C. to 37° C., preferably 5° C. to 10° C., for 0.5 to 24 hours in darkness.

The molar amount of the activation agent is in the range of 0.03 to 0.3 molar equivalents with respect to hyaluronan dimer.

The solvent used in the activation reaction is selected from the group comprising isopropanol, tert-butanol, dioxane, and tetrahydrofuran.

The activation agent is benzoyl chloride, the organic base is selected from a group comprising N,N-diisopropylethylamine, triethylamine, trimethylamine, dimethylaminopyridine, preferably trimethylamine and the solvent is selected from a group comprising isopropanol, tert-butanol, tetrahydrofuran (THF), dioxane, isopropanol.

In one preferred aspect of the present embodiments, the conjugate of HA−ATRA is produced by the process comprising

(a) reacting the antioxidant such as all-trans retinoic acid (ATRA) with benzoyl chloride (Scheme I).

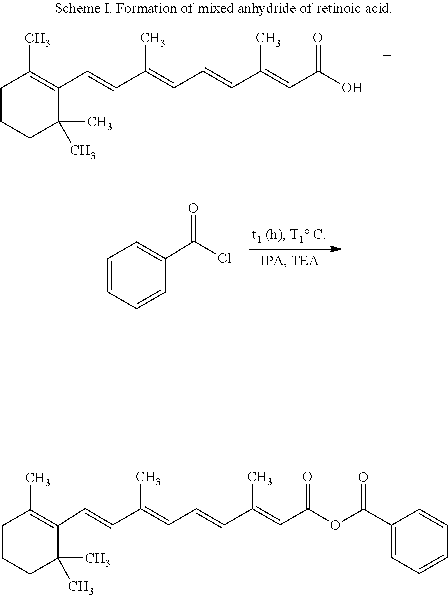 [SMILES: CC1=C(/C=C/C(C)=C/C=C/C(C)=C/C(=O)O)C(C)(C)CCC1 ; O=C(Cl)c1ccccc1 ; CC1=C(/C=C/C(C)=C/C=C/C(C)=C/C(=O)OC(=O)c2ccccc2)C(C)(C)CCC1]

(b) reacting hyaluronan with a mixed anhydride to produce a covalent bond between the antioxidant and hyaluronan, wherein the antioxidant possesses at least one group capable of reacting with hydroxyl residue present in the skeletal to produce a covalent bond between the antioxidant and the polymer. An exemplary procedure for producing modified hyaluronan using ATRA as the antioxidant to link (Scheme II).

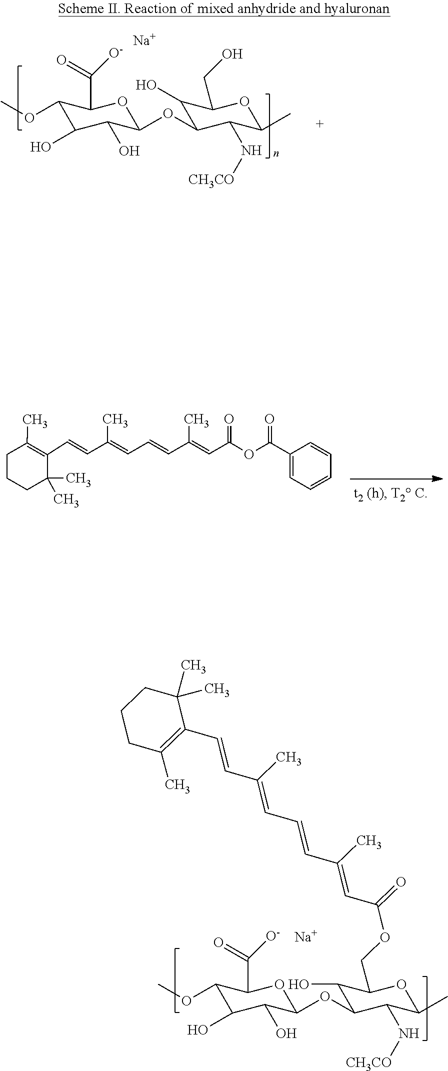

However, this reaction can be further used for the activation of any carboxylic acid moiety of antioxidants described in the art such as gallic acid, ferulic acid, caffeic acid, hydrocaffeic acid and many antioxidants previously described in the art.

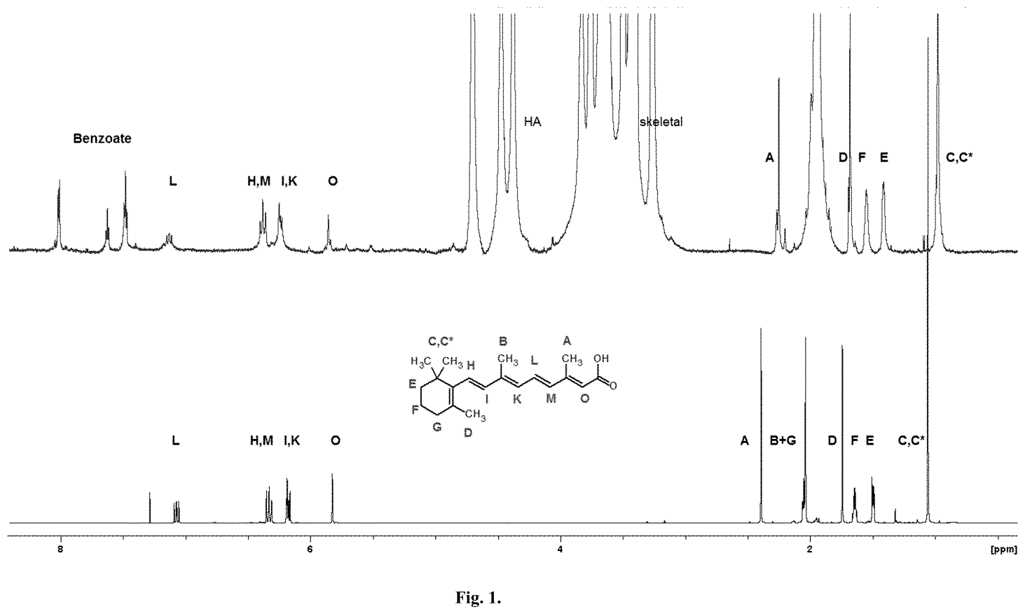

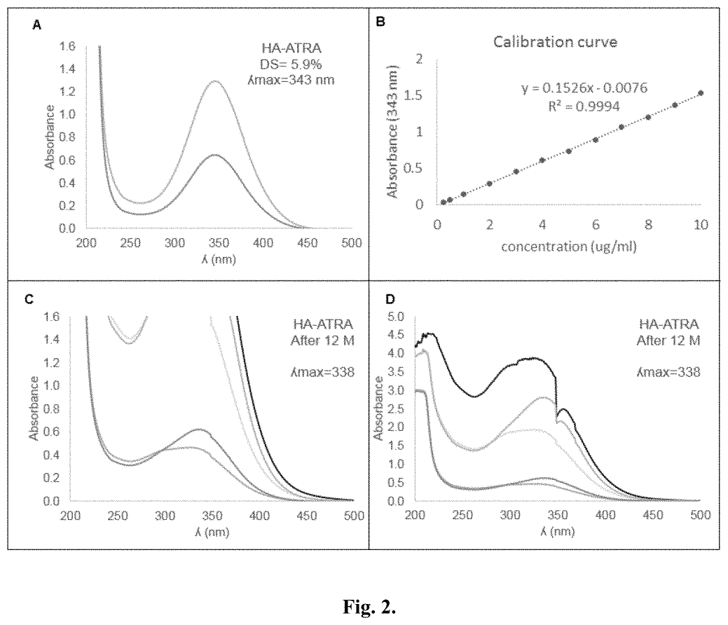

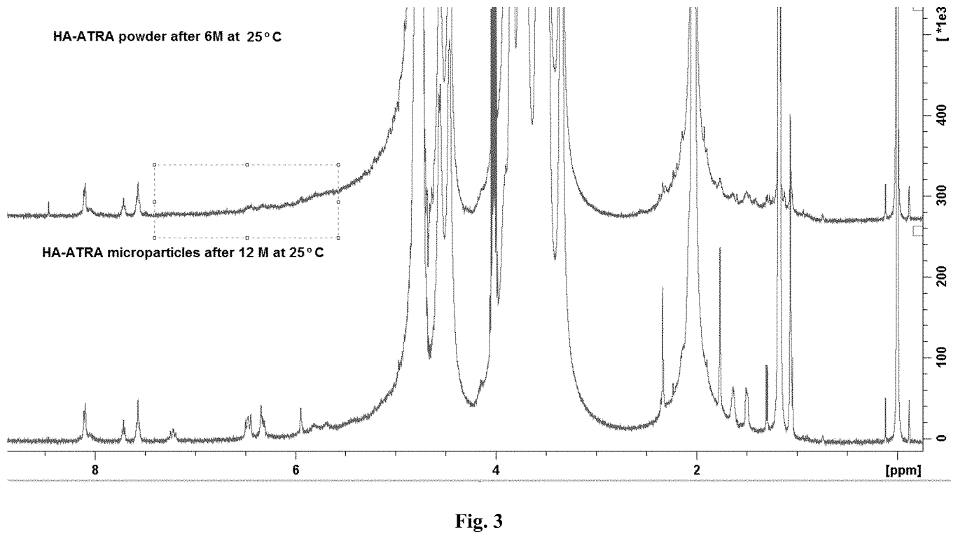

In this disclosure, the identification of the chemical structure of the modified polysaccharide as well as the determination of the degree of substitution can be performed by NMR (FIG. 1). However, as NMR is imprecise for determination if such a low degree of modification, the hydrolysis of the retinoic ester is preferred and the determination of the degree of substitution is carried out by UV-vis as people skilled in the art are familiar (FIG. 2).

The further embodiment of the present embodiments comprises the method of the production of microparticles according to the present embodiments that comprises several steps. The first step of the production is a preparation of a mixed anhydride of retinoic acid that is carried out by benzoyl chloride (see Scheme I above), in the presence of an organic solvent miscible with water with high dielectric constant. The preferred solvents used in the reaction are isopropanol, tert-butanol, THF or dioxane. The temperature of the activation is crucial for the formation of the intermediate. The reaction is carried out at low temperature or temperature up to room temperature (0 to 25° C.) and for a time span ranging from 5 to 30 minutes. Surprisingly, benzoyl chloride does not cause isomerization or degradation of retinoic acid during the reaction, as compared to the use of 3-[3-methylamino)propyl]-1-ethylcarbodiimide (EDC) hydrochloride as activating agent (of ATRA) that led to a concomitant isomerization of the double bonds in the molecule (see Christensen, M. S., Pedersen, P. J., Andresen, T. L., Madsen, R. and Clausen, M. H. (2010), Isomerization of all-(E)-Retinoic Acid Mediated by Carbodiimide Activation—Synthesis of ATRA Ether Lipid Conjugates. Eur. J. Org. Chem., 2010: 719-724. doi:10.1002/ejoc.200901128). The isomerization of the double bond starts the degradation of the retinoid, also lower yield is expected due to the formation of side products. Furthermore, FIG. 1 shows that the signal 0 did not appear as a doubled signal as described by Christensen as a clear signal of isomerization of the retinoyl moiety.

The second step of the production is the reaction of hyaluronan with the mixed anhydride at low temperature (from 0 to 25° C.) (see Scheme II above). A considerable advantage to previously reported art is that the polysaccharide is directly solubilized in water without the use of any acid catalyst, which may induce the degradation of the polysaccharide. The esterification reaction is kept under constant stirring for 1-5 hours, even preferable for 3 h. The use of this reaction is selective and allows for the final esterification products characterized by the fact that the hydroxyl groups of HA that have been esterified with retinoic acid. In this case, the reaction presents a considerable advantage to the previously reported art, U.S. Pat. No. 6,897,203, which clearly stated that HA is suspended in an organic solvent under stirring for 1-5 hours at 25-100° C., which clearly degrades the polysaccharide due to the combination of acid conditions and high temperature and prolonged reaction time (17 h).

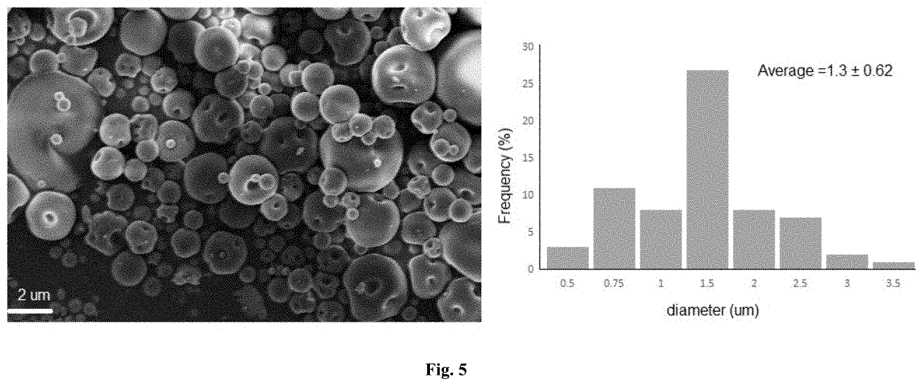

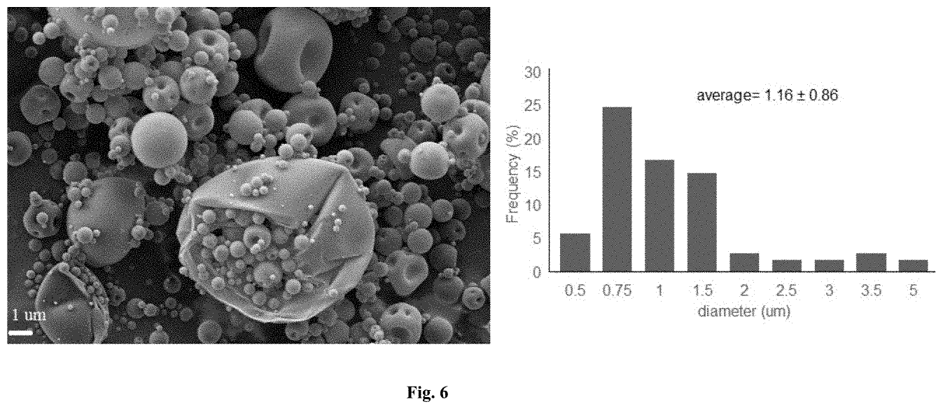

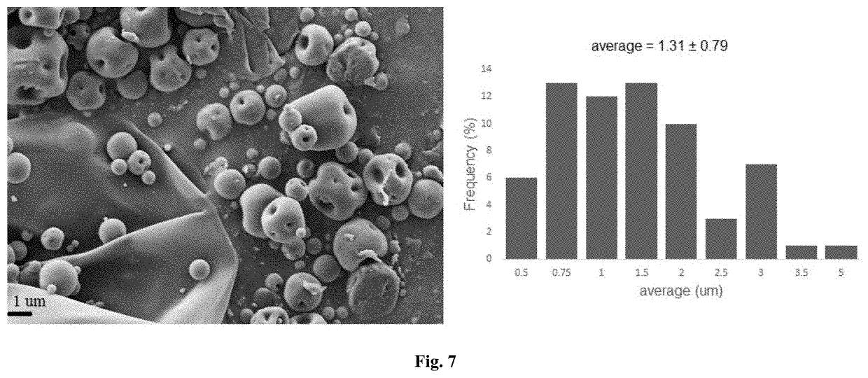

The third step of the method of the production of microparticles of HA−ATRA conjugates is processing techniques helpful for the preparation of polymeric microparticles. Particularly, spray-drying is a useful technique. Spray-drying is a well-established method used in the industry for producing microparticles or microencapsulates after a solubilized polymer, which is then atomized into droplets, and brought into contact with a hot process gas. However, the way of processing is not limited to spray drying but to any technique that produces micro and nanoparticles characterized by small size and narrow size distribution. As people skilled in the art knows, spray drying can be modulated giving small microparticles of size up from 100 nm and up to 10 μm [Sosnik A, Seremeta K P. Advantages and challenges of the spray-drying technology for the production of pure drug particles and drug-loaded polymeric carriers. Adv Colloid Interface Sci 2015; 223:40-54.]. Particularly the formation of microparticles with a diameter characterized by 1.3±0.8 μm (see FIGS. 5 to 7) were obtained in this disclosure. The third step of the method is performed. Particularly at the inlet temperature of between 100° C. to 200° C., and the outlet temperature between 80° C. to 100° C. More particularly 180° C. (inlet) and 90° C. (outlet).

Particularly, this process of drying led to the formation of a stable composition in the form of microparticles.

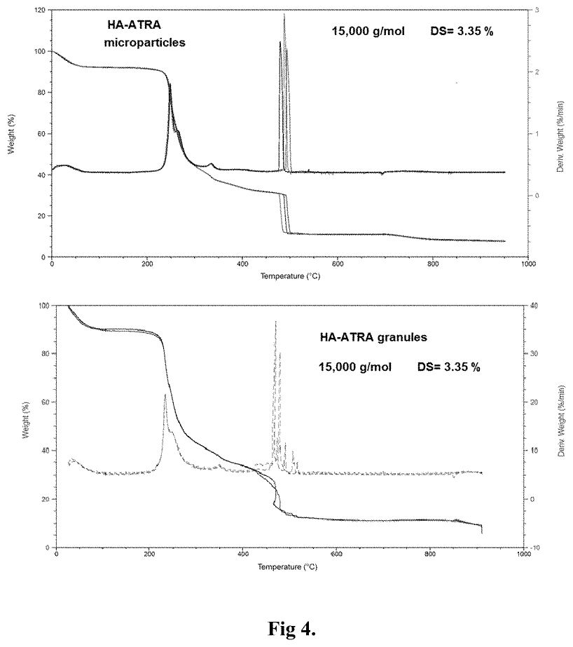

Surprisingly, the thermogravimetric analysis (TGA) of HA−ATRA granules (see FIG. 4b) shows that ATRA is unstable even after preparation. Moreover, ATRA will present additional degradation after been maintained at 25° C. for prolonged time (Table 1, FIG. 2c).

Furthermore, the chemical characterization of the microparticles obtained after processing the conjugate of HA−ATRA by spray-drying was performed by means of UV-Vis spectroscopy. FIG. 2a shows the absorption maxima (λmax) corresponding to microparticles made of the conjugate of HA−ATRA. Moreover, this maximum was used to detect possible changes on the structure of HA after processing by spray-drying and to quantify the amount of retinoate esters of HA found on the microparticles by using a calibration curve. Surprisingly, HA−ATRA (granules) suffers changes after storage. Both a hyperchromic effect due to cross-linking of the molecule (FIGS. 2c and 2d). It became evident from somebody skilled in the art that, a hypsochromic shifting was produced due to loss of conjugation (FIG. 2d). Additionally, ¹H NMR is also showing that granules obtained after precipitation of HA−ATRA are not stable after 6 M of storage at room temperature (FIG. 3). The degradation was confirmed by TGA analyses (FIG. 4), with the presence of many products ˜500° C.

The chemical conjugation of ATRA to HA protected the retinoid from degradation. Obviously, by yielding a half-life (greater than free retinoic acid and/or retinoids).

In this disclosure, it is further reported that the amount of active ATRA is conserved after four weeks of storage at 40° C., in the absence of any further toxic antioxidant, which is an advantage to the previously reported art such as US 20190015366 A1 and WO2015092602A1, that require the presence of the toxic benzoyl peroxide, which have been reported as an inductor of photo-carcinogenesis in hairless mice after solar radiation. Similar art was reported in US20100166852A1. In the present embodiments, the microparticles made of HA−ATRA were stored for prolonged times in the presence of air the microparticles are stable, while the obtained powders degraded faster (FIGS. 2 and 4). Surprisingly, a combination of high degree of substitution and high temperature i.e. 25° C. the particles degrade. In this disclosure, the thermal decomposition of the HA−ATRA microparticles was compared with the native polysaccharide and pure retinoic acid using thermogravimetric analysis (TGA) according to de Mendonça CMS, de Barros Lima IP, Aragão CFS, Gomes APB in Thermal compatibility between hydroquinone and retinoic acid in pharmaceutical formulations. Journal of Thermal Analysis and calorimetry 2014; 115:2277-85.

The results, including initial temperature at which thermal decompositions starts at Tₒₙₛₑₜ=222.58° C. for HA, while HA−ATRA presented Tₒₙₛₑₜ=227.32° C. The TG curve of ATRA shows only three stages of decomposition in the temperature range of 185−609° C. First, the results clearly demonstrated that chemical modification of HA led to higher thermal stability. Second, the conjugate HA−ATRA had higher thermal stability than native HA. It becomes obvious for a person skilled in the art that only the physical mixture of ATRA decreased even more the stability.

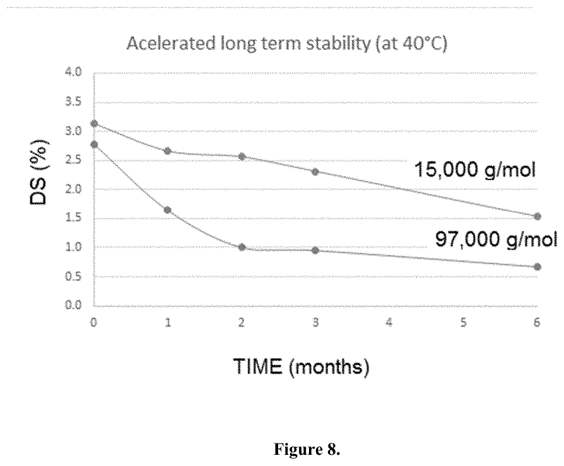

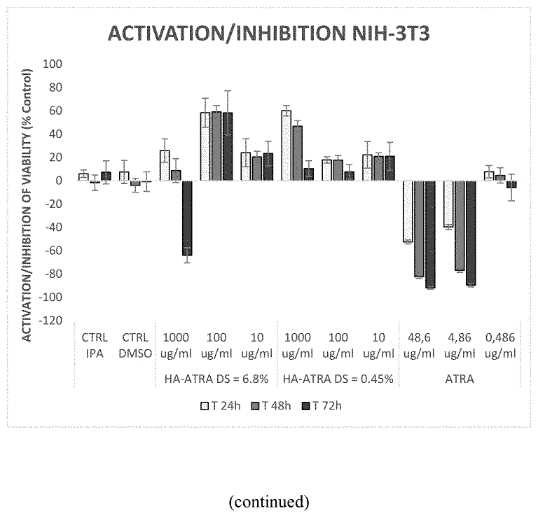

In conclusion, an efficient combination of degree of substitution (up to 2.9%) and molecular weight (preferably low, due to the lower degradation rate of the polymer during long term stability (Mw from 10,000 and up to 30,000 g/mol) makes the microparticles made of HA-ATRA stable (FIG. 8). Surprisingly, the use of spray-drying, which involve high temperature does not change the biological activity of the conjugate and even its Mw.

The microparticles according to the present embodiments are long-term thermostable when the degree of substitution in the conjugate of hyaluronan of the general formula is in the range from 0.5% to 8%, preferably 0.5% to 6.5% and when the molar weight of the conjugate of the general formula I is in the range from 6,000 g/mol to 30,000 g/mol, preferably from 6,000 g/mol to 20,000 g/mol.

Furthermore microparticles according to the present embodiments are long-term thermostable, at least 12 months at the temperature from 20° C. to 40° C., preferably from 20° C. to 30° C., more preferably from 20° C. to 25° C., the most preferably at 25° C. when the degree of substitution in the conjugate of hyaluronan of the general formula is in the range from 0.3% to 3.1%, preferably from 0.3% to 2.5% and when the molar weight of the conjugate of the general formula I is in the range from 6,000 g/mol to 30,000 g/mol, preferably from 6,000 g/mol to 20,000 g/mol.

Another aspect of the present invention is a composition comprising microparticles of a conjugate of all-trans retinoic acid and hyaluronan of the present invention containing the conjugate of all-trans retinoic acid and hyaluronan of the general formula I as defined above. The amount of the conjugate is in the range of 0.001 to 20 wt. %, preferably 0.005 to 10 wt. %, more preferably 0.01 to 5 wt. %, the most preferably 0.1 to 0.5 wt % by the weight of the composition. The conjugate of HA−ATRA concentration is preferably greater than 0.01% by weight, e.g., at least about 0.1% by weight, and more preferably at least about 0.05% by weight HA−ATRA in the vehicle. Concentrations greater than 0.5% by weight are unnecessary and not preferred. A particularly preferred formulation contains about 0.1% by weight in a liquid carrier comprising water and/or water containing polyethylenglycol (PEG) 400,000 g/mol. These concentrations of HA−ATRA are reported as percent by weight.

The microparticles according to the present invention containing the conjugate of HA-ATRA can be presented in emulgated form, suspended form, dissolved form, the dispersed form or as rehydrated microparticles in the composition according to the present embodiments. The form of composition according to the present embodiments, preferably the cosmetic composition, can be selected from a group comprising suspension, emulsion, dispersion, solution. The preferred embodiment of the present embodiments is the cosmetic composition, such as face cream formulation wherein (a) from 0.001 to 0.1% by weight of active ingredient or HA−ATRA conjugate, further it can comprise (b) 6.0 to 32.0% by weight of cosmetically acceptable additives selected from a group comprising:

(i) at least one fat selected from the group comprising of natural, modified or synthetic fatty acids or its derivative, selected from a group comprising sorbitan monostearate, glyceryl stearate, PEG-100 stearate, stearic acid, caprylic/capric triglyceride,
(ii) at least one nonionic surfactant and emulsifier, selected from a group comprising polysorbate, polysorbate-60, cetearyl polyglycoside,
(iii) at least one oil or vegetable extract or fats, selected from a group comprising shea butter, cocoa butter, jojoba oil, avocado oil, especially hydrogenated avocado oil
(iv) at least one alcohol selected from a group comprising cetyl alcohol, benzylalcohol, and (v) at least one moisturizer selected from a group comprising glycerin, propylene glycol, butylene glycol;
and
(c) addition of hydrophilic gel-cream base or water to the q.s.p. (quantitié suffisante pour) 100% by weight of the composition. It means that amount of hydrophilic gel-cream base or water is in the range of 67.9 to 93.9% by weight of the composition. Components of the hydrophilic gel-cream base are well known for a person skilled in the art. They can be selected from a group comprising Cetomacrogol emulsifying wax (BP), paraffin, propylene glycol, water.

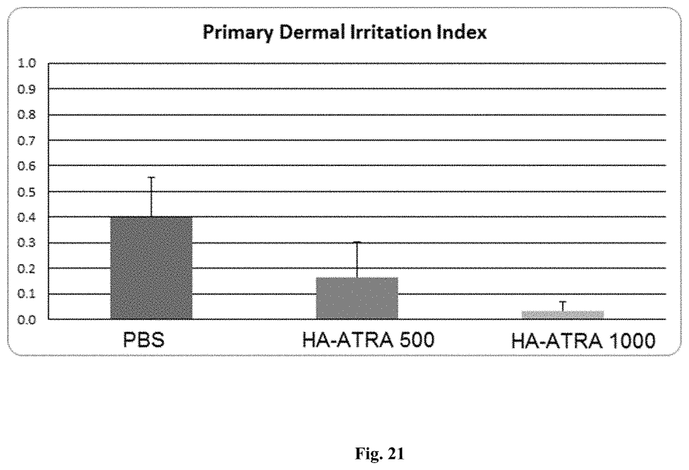

Components used in the cosmetic compositions according to the present embodiments are known in the art and they are available and generally used in the various formulations known or available in the art, including creams, dressings, gels, hydrogels, ointments and liquid polymers, including hyaluronan or amphiphilic hyaluronan derivatives. The HA−ATRA microparticles in the vehicle is such that the topical application won't cause desquamation of the skin, including superficial and/or subclinical peeling (example 28, FIG. 21).

Furthermore, the topical aqueous composition of the microparticles of this disclosure can be further mixed with any hydrophilic polymer such as hyaluronan or cross-linked polymer in an amount of about 1% to about 75% by weight, preferably 0.5 to 10% by weight of the composition to form a gel, which can be applied in the skin. The crossed-linked polymer can be selected from a group comprising oxidized HA, aminated HA or a polymer able to form a Shiff base. It became obvious for somebody skilled in the art that a gel can be used as reservoir (WO2018122344A1 and US20180071193A1). However, the compositions need an additional antioxidant as benzoyl peroxide. The method of preparing a topical aqueous composition comprising the water-soluble microparticles made of HA−ATRA is dispersing the material in water without the use of a surfactant; which is an advantage to previously reported art US 20100029765. The pH is adjusted to about 4 to about 6.5.

The composition comprising microparticles of the conjugate of all-trans retinoic acid and hyaluronan of the present invention contains at least one hydrophobic compound encapsulated by the conjugate of all-trans retinoic acid and hyaluronan. The hydrophobic compounds are selected from a group comprising bioactive compounds such as vitamins or antioxidants, such as resveratrol, curcumin, retinyl palmitate, vitamin E. The amount of the hydrophobic compound is in the range from 1 to 3% by weight of the composition. The microparticles containing conjugate of HA−ATRA can be rehydrated (see Examples 32-35).

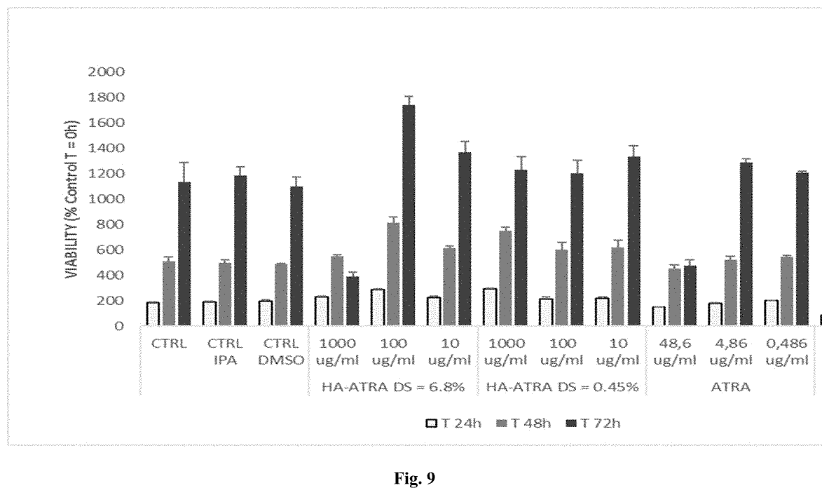

Moreover, ATRA in higher doses is known to be cytotoxic. However, conjugation of ATRA and HA mitigated acute cytotoxicity (FIG. 9). A very important advantage of the present embodiments is that the toxic effects of ATRA are attenuated due to the presence of HA. Oppositely, Castleberry et al reported the formation of nanofibular nanoparticle polymer-drug conjugate for sustained dermal delivery of retinoids includes the conjugation of ATRA to PVA using the Steglich esterification process mediated via DCC (N, N′-dicyclohexycarbodiimide) chemistry. In the case of ATRA conjugated to PVA (PATRA) a similar decrease in proliferation was observed (US2018185513 (A1)/WO2016210087A1). Unfortunately, the fate and degradation mechanism of PVA are still unknown and the incidence of long-term adverse reactions secondary to the injection of a foreign material (PVA) are still ignored. Furthermore, the conjugation to amphiphilic block consisting amine-based compounds (KR2017142961A).

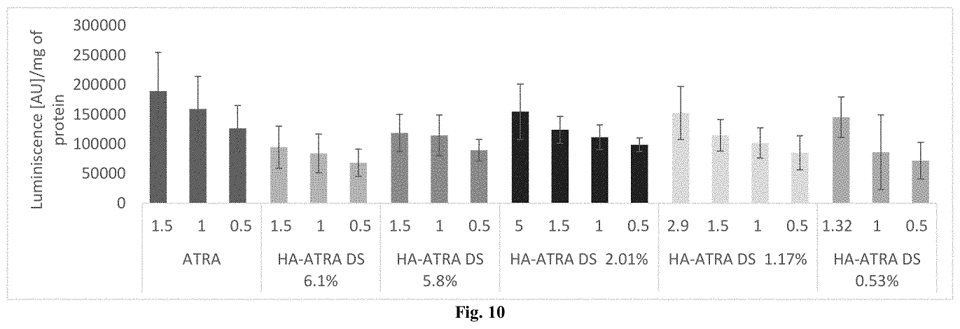

The presented HA−ATRA microparticles according to the present embodiments retained the abilities of unbound ATRA and/or retinoids to induce gene expression via mechanisms of binding to specific DNA elements (FIG. 10). Particularly, the microparticles made of HA−ATRA were able to induce expression of cholesterol metabolism genes. As people skilled in the art assume, the molecule of cholesterol is an essential structural component of the vertebrate cell membrane as well as a precursor of steroid hormones, vitamins, and bile acids (Zhang D, Tomisato W, Su L, Sun L, Choi J H, Zhang Z, et al. Skin-specific regulation of SREBP processing and lipid biosynthesis by glycerol kinase 5. Proc Natl Acad Sci USA 2017; 114:E5197-E206.). The biosynthesis of cholesterol and other lipids in the skin is essential for the formation of new epidermal permeability barrier in aged skin, for hair follicle morphogenesis and maintenance. The induction of cholesterol metabolism is beneficial for maintaining the skin barrier. As it is well known by people skilled in the art that skin barrier health and cholesterol content decreases as a function of age, resulting in a thinning of the barrier, greater water loss, dryness, and increased permeability to toxins and free radicals. Age-related changes in skin also enhance transepidermal water loss (TEWL), which can be counteracted with induction of cholesterol synthesis. On the other hand, inhibition of cholesterol synthesis with statins leads to increased TEWL. Retinoids are known to increase TEWL. ATRA may decrease cholesterol metabolism. Keratinocytes treated with ATRA had lower gene expression of cholesterol metabolism genes. Cholesterol content in the cells is regulated also by its efflux from cells via ABCA1 transporter. Surprisingly, unbound (or free) ATRA did not affect cholesterol metabolism via the expression of ABCA1, on the contrary, while 9-cis retinoic acid decreased cellular cholesterol via ABCA1 increased expression. Also, ATRA treatment decreased total cholesterol content in monocytes.

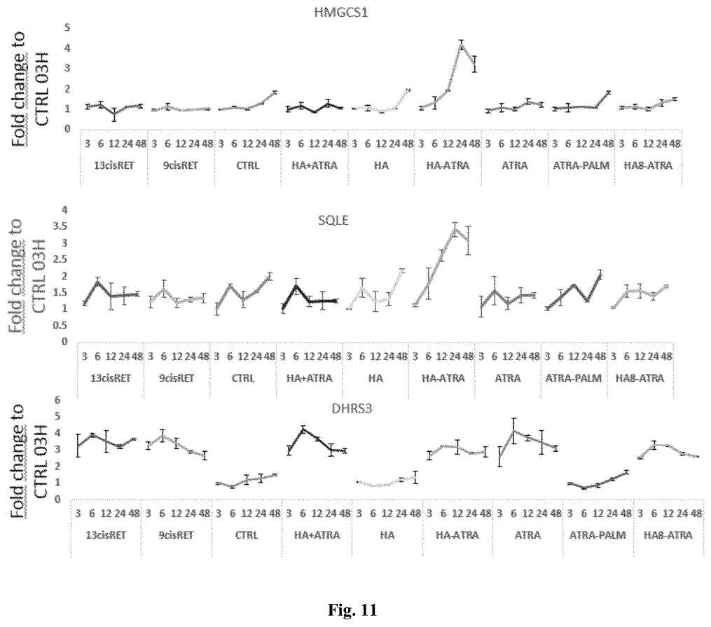

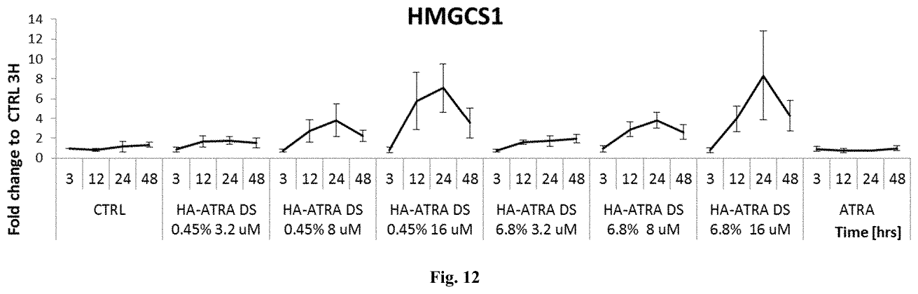

As people skilled in the art know, one of the mechanisms of cellular regulation of cholesterol synthesis is a coordinated gene expression of the cholesterol synthesizing enzymes such as (HMGCS1, 3-Hydroxy-3-Methylglutaryl-CoA Synthase 1 and SQLE, Squalene Epoxidase. When keratinocytes or fibroblasts were treated with the microparticles made thereof. Surprisingly, the gene expression of the cholesterol metabolism in the genes HMGCS1 and SQLE was induced during the assayed time (48 hours), as presented in FIG. 11. Particularly, this effect is specific to the HA−ATRA conjugate. Both, unbound ATRA or a physical mixture with HA (in other words when ATRA was not covalently conjugated) did not induce gene expression of HMGCS1 and SQLE. In addition, neither unbound 9-cis retinoic nor 13-cis retinoic acid were able to upregulate HMGCS1 nor SQLE. This implies that the microparticles according to the present embodiments induce gene expression of similar targets and increased cholesterol metabolism. Thus, they overcome the known drawbacks of retinoids on TEWL and cholesterol synthesis. Furthermore, FIG. 12 shows that the expression of HMGCS1 in fibroblasts after treatment with the microparticles described in Example 15, in which concentration corresponds to micromoles of added retinoic acid. It is evident that the effect on HMGCS1 expression can be reached by the microparticles in concentration of active ATRA of 5 to 100 μg/mL. The advantage of the microparticles containing HA−ATRA conjugates according to the present invention is its simplicity and yet unique activity.

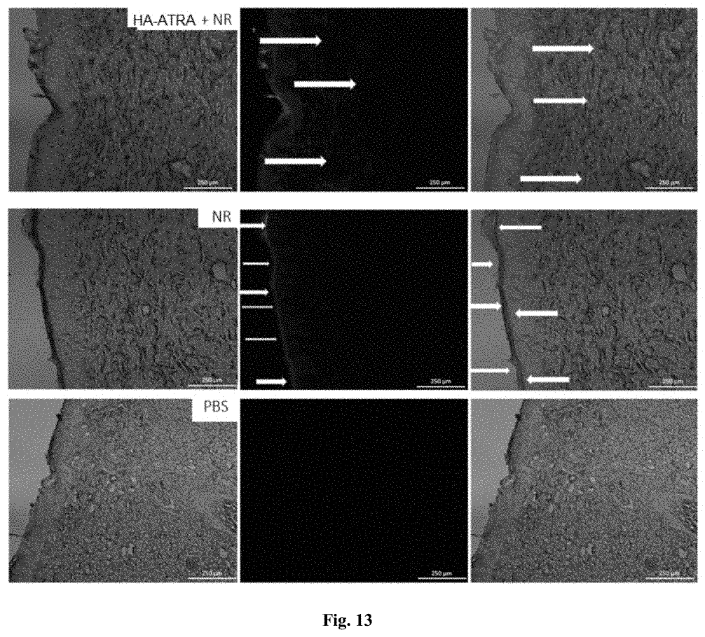

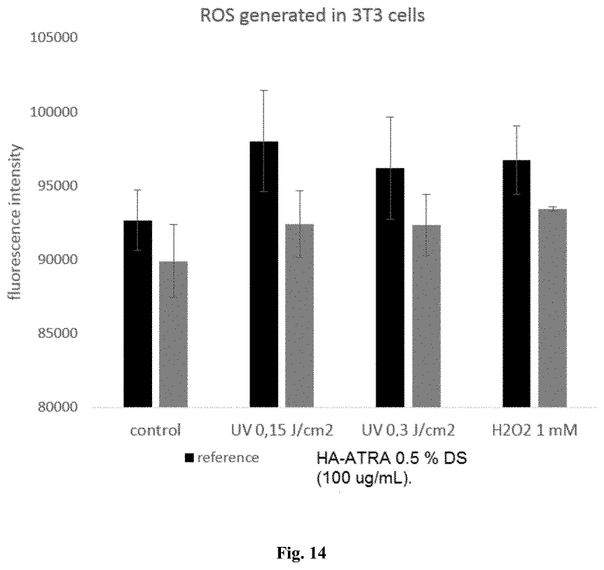

Due to its natural presence in skin, and its depletion during aging, exposure to UV radiation (sunburns and photoaging), and other skin trauma, HA is also included in many skin products in addition to its use as an injectable filler. Topically applied HA must gain entry through the hydrophobic layer of ceramide/keratin covering the outer layers of keratinocytes. However, the skin penetration is rather complicated due to the lipid-rich stratum corneum present on the skin surface. Moreover, HA, a polyanion, is not expected efficiently to cross the skin's keratinocyte layer. Therefore, topical HA either remains a surface treatment (e.g., HA-containing creams) or is injected if significant penetration into the skin is desired (e.g., in the treatment of wrinkles). In this case, the ability to penetrate deeper into the tissues is a major benefit for agent's topical functionality. The amphiphilic nature of the HA−ATRA conjugate and ability to encapsulate hydrophobic compounds (examples 32-35 or Nile red on Example 21). The last example was utilized as model to demonstrate the skin penetration of the composition made thereof. The more pronounced fluorescence in the both epidermis and dermis of Nile red encapsulated in our HA-ATRA conjugate in comparison to free Nile red is a direct indicator of the composition ability to penetrated through stratum corneum and basal lamina on epidermal-dermal junction and ability to exert its biological functions in both epidermal keratinocytes and dermal fibroblasts (FIG. 13).

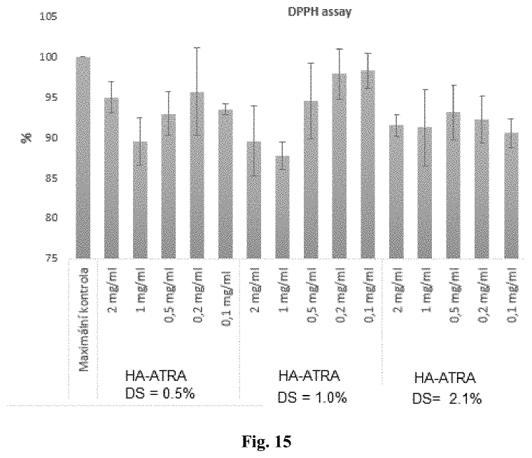

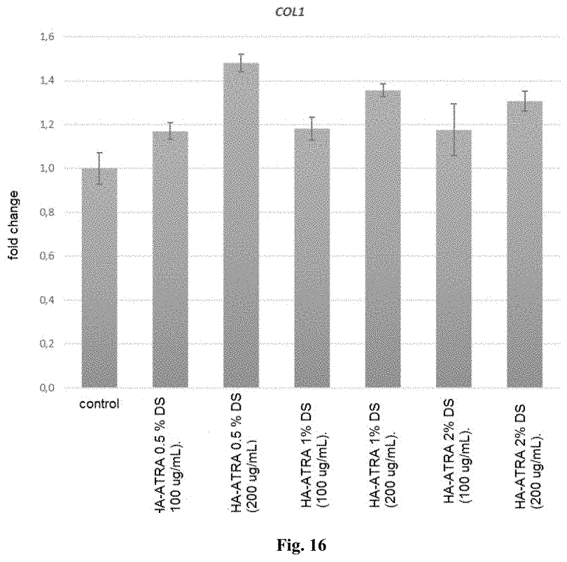

A further object of the present invention is to provide a composition suitable for use in dermal enhancement, hyaluronan replenishment and/or protection therapy against the signs of aging of the skin and/or various forms of skin atrophy. According to this disclosure, cells incubated with microparticles made of HA−ATRA generated less reactive oxygen species (ROS) in comparison with the control (cells incubated in Normal Human Dermal Fibroblasts medium (NHDF medium)) (FIG. 14). These findings were confirmed using DPPH (2,2-diphenyl-1-picryl-hydrazyl-hydrate) assay—an acellular test for evaluation of radical scavenging activity, measured calorimetrically (on FIG. 15). The result showed that in the presence of HA−ATRA microparticles, there is less free radicals (in comparison with a control).

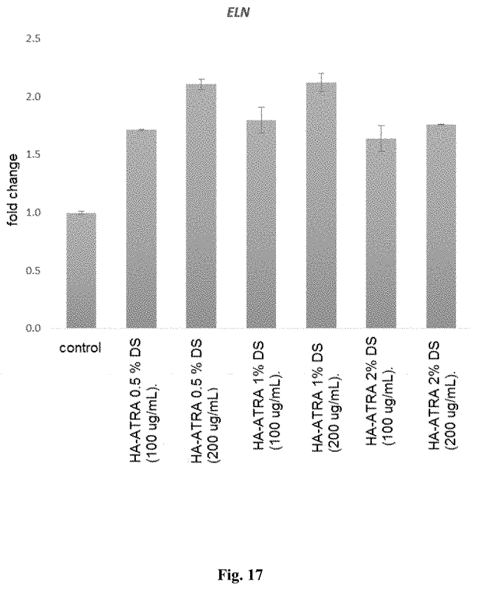

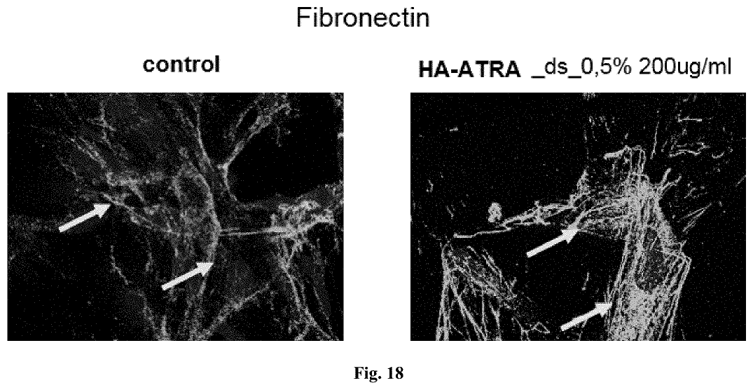

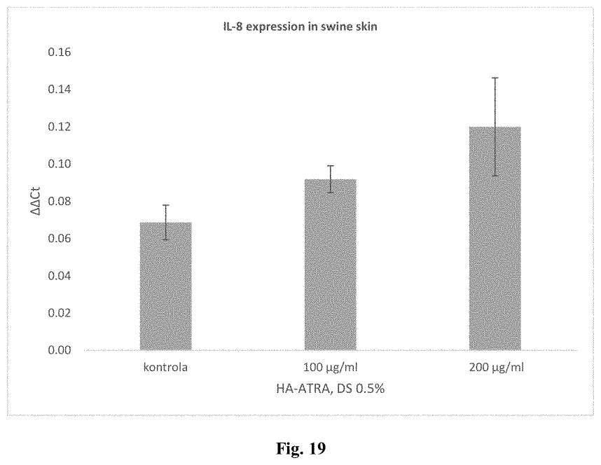

In another aspect of the present application, the treatment with the microparticles made of HA−ATRA (DS=0.5%) caused an induction of COL1A gene expression in WS1 human fibroblasts (FIG. 16). Particularly, the use of the microparticles in any cosmetic composition will increase collagen production. Furthermore, the microparticles induce expression of elastin (FIG. 17) and fibronectin (FIG. 18). Together these results demonstrate the anti-ageing properties of HA−ATRA microparticles. FIG. 19 demonstrated the significant induction of IL-8 (Interleukin 8) after incubation of swine skin with microparticles HA−ATRA. IL-8 is connected to stimulation of angiogenesis and skin regeneration.

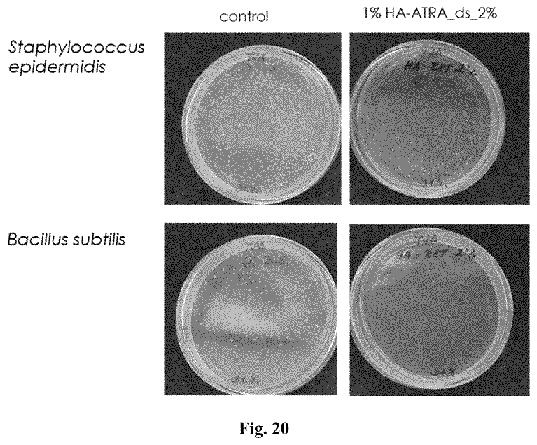

FIG. 20 demonstrated that the microparticles made of the HA−ATRA conjugate demonstrated antimicrobial activity for Bacillus subtilis and Staphylococcus epidermidis, which is involved during the development of Rosacea. Similar activity was previously observed for retinaldehyde (RAL), however, Pechere et al believed that RAL activity is likely due to the aldehyde group in the isoprenoic lateral chain and this structural characteristic differs from parent natural retinoids such as retinol (ROL) and ATRA [Pechere M, Germanier L, Siegenthaler G, Pechere J C, Saurat J H. The antibacterial activity of topical retinoids: the case of retinaldehyde. Dermatology 2002; 205:153-8]. Obviously, the aldehyde moiety is also absent in the HA−ATRA conjugate of the present invention.

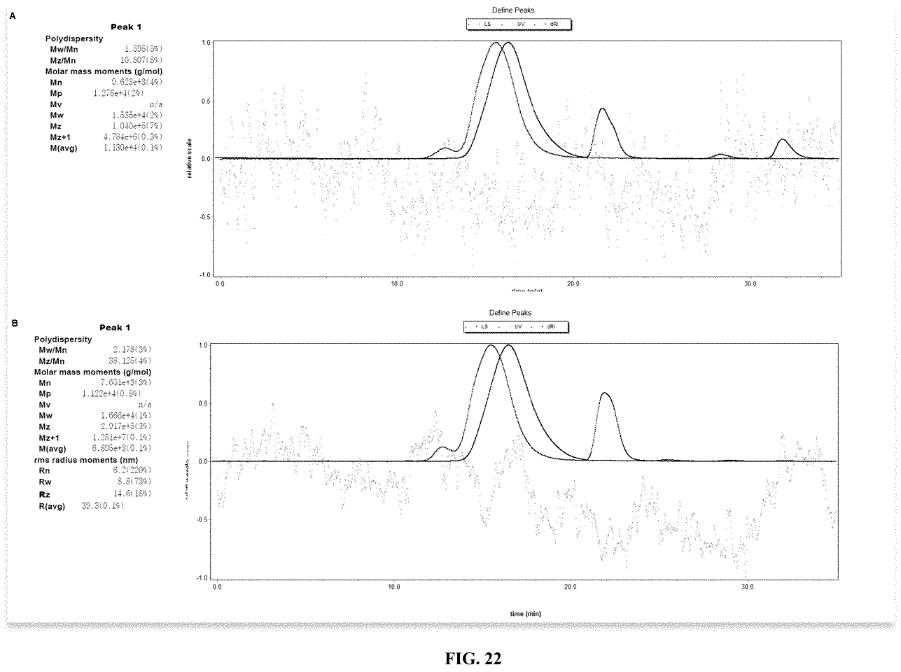

There is not Mw loss of the conjugate in microparticles according to the present embodiments after spray-drying and even long-term storage as it can be seen from FIG. 22.

Another aspect of the present invention the microparticles or the composition according to the present invention can be used in cosmetics or in medicinal applications for improving epidermal barrier maintenance in skin, that transcriptionally regulates lipid synthesis, specifically cholesterol synthesis.

They are used especially as anti-aging agent to induce induces collagen 1, fibronectin or elastin expression and as an antimicrobial agent effective against Gram-positive bacteria, preferably selected from a group comprising Bacillus subtilis, Staphylococcus epidermidis.

This research was supported by the European Regional Development Fund—Project INBIO (No. CZ.02.1.01/0.0/0.0/16_026/0008451).

### Definitions of the Terms

In this disclosure the term, “hyaluronic acid” or “hyaluronan” or (HA) is a lineal polysaccharide composed of this repeating unit: (1→3)-β-N-acetyl-D-glucosamine-(1→4)-β-D-glucuronic acid.

The term “pharmaceutically acceptable salt” as used herein, are preferably ions of alkali metals or ions of alkaline-earth metals, more preferably Na⁺, K⁺, Mg²⁺ or Li⁺.

The term “retinoic acid” refers to the molecule identified as retinoic acid, i.e. 3,7-dimethyl-9-(2,6,6-trimethyl-1-cyclohexene-1-yl)-2,4,6,8-nonatetraenoic acid, thus it is further identified as ATRA (All trans-retinoic acid).

The term “degree of substitution” or “(DS)” indicates the (average) number of the residue of all-trans retinoic acid of the formula II per 100 hyaluronan dimer.

The term “granules” are entities in which primary powders adhere, so that means a dry, bulk solid composed of many fine particles, wherein more of 97% of particles have an average granule size between 1 to 5 mm.

The term “microparticles” means that the material contains mono particles between 500 nm to 5 μm in average size.

The term “room temperature” defines it as being simply 15 to 25° C.

### EXAMPLES

### Example 1. Synthesis, Purification and Isolation and Preparation of Microparticles Containing Retinoic Acid Attached to HA (HA−ATRA)

Hyaluronic acid (2.0 g, 5 mmol) characterized by an average molecular weight of 15,000 g/mol was dissolved in 40 mL of distilled water. To that solution, 20 mL of isopropanol (IPA) was added. After the solution was homogeneous, triethylamine (1.395 mL, 2.5 mmol) and DMAP (31.5 mg, 0.031 mmol) were consequently added to the mixture under stirring. In a second reaction flask, 0.045 mg of retinoic acid (0.2 mmol, 0.03 eq to HA dimer) were dissolved in isopropanol (5 ml) and activated by 0.004 ml of benzoyl chloride (0.2 mmol, 0.03 eq to HA dimer) in the presence of 1.395 mL of triethylamine (TEA). The activation was carried out for 60 minutes at 5° C. in darkness, after that time the activated mixture was added to solution containing HA. The resulting solution was maintained at 5° C. for 3 h in darkness. A saturated solution of sodium chloride was added to the reaction to precipitate the polymer. After that, the polymer was washed with an excess of anhydrous IPA (50 mL). The product was washed four times with solutions of isopropanol: water 85% (v/v) (4×50 mL). Finally, the precipitate was washed two more times with isopropanol. The product was filtrated and solubilized in water in a final concentration of 0.5% (w/v). Finally, the product was spray-dried using a mini spray dryer Büchi Mini Spray Drier B-290, which operates in a co-current mode and is equipped with a 0.7 mm diameter two-fluid nozzle. (inlet temperature: 190° C.; outlet temperature 90° C., solution feed rate: 10 mL/min, atomization air flow rate of 0.5 kg/h in a spray chamber size 165 mm/600 mm. Powder samples were stored in closed sachets at room temperature immediately after spray-drying to limit moisture uptake of the samples between production and testing. The particle size distribution of the solid was measured by Scanning Electronic Microscopy (SEM). (Average size of the batch=1.5 (±) 0.5. μm)

Additionally, the concentration of ATRA in the polymer was determined by UV-Vis. For that experiments, retinoic acid used for the chemical modification was dissolved in basic media, consisting of sodium hydroxide, sodium hydrogen carbonate or sodium bicarbonate mixed with isopropanol. This solution was used to create the calibration curve depicted in FIG. 1B using the equation showed in FIG. 1B, the amount of ATRA in the polymer was calculated by dissolving HA−ATRA in the same media and reading the Amax at 343 nm. Each sample was measured in triplicate.

The amount of ATRA found in the sample is considered as 0.65 wt %
Degree of substitution determined by NMR (DS)=0.5%.

### Example 2. Synthesis, Purification and Isolation and Preparation of Microparticles Containing Retinoic Acid Attached to HA (HA−ATRA)

Hyaluronic acid (2.0 g, 5 mmol) characterized by an average molecular weight of 6,000 g/mol was dissolved in 40 mL of distilled water. To that solution, 20 mL of isopropanol (IPA) was added. After the solution was homogeneous, triethylamine (1.395 mL, 2.5 mmol) and DMAP (31.5 mg, 0.031 mmol) were consequently added to the mixture under stirring. In a second reaction flask, 0.045 mg of retinoic acid (0.2 mmol, 0.03 eq to HA dimer) were dissolved in isopropanol (5 ml) and activated by 0.004 ml of benzoyl chloride (0.2 mmol, 0.03 eq to HA dimer) in the presence of 1.395 mL of triethylamine (TEA). The activation was carried out for 60 minutes at 5° C. in darkness, after that time the activated mixture was added to solution containing HA. The resulting solution was maintained at 5° C. for 3 h in darkness. A saturated solution of sodium chloride was added to the reaction to precipitate the polymer. After that, the polymer was washed with an excess of anhydrous IPA (50 mL). The product was washed four times with solutions of isopropanol: water 85% (v/v) (4×50 mL). Finally, the precipitate was washed two more times with isopropanol. The product was filtrated and solubilized in water in a final concentration of 0.5% (w/v). Finally, the product was spray-dried using a mini spray dryer Büchi Mini Spray Drier B-290, which operates in a co-current mode and is equipped with a 0.7 mm diameter two-fluid nozzle. (inlet temperature: 190° C.; outlet temperature 90° C., solution feed rate: 10 mL/min, atomization air flow rate of 0.5 kg/h in a spray chamber size 165 mm/600 mm. Powder samples were stored in closed sachets at room temperature immediately after spray-drying to limit moisture uptake of the samples between production and testing. The particle size distribution of the solid was measured by Scanning Electronic Microscopy (SEM). (Average size of the batch=1.5 (±) 0.5. μm)

Additionally, the concentration of ATRA in the polymer was determined by UV-Vis. For that experiments, retinoic acid used for the chemical modification was dissolved in basic media, consisting of sodium hydroxide, sodium hydrogen carbonate or sodium bicarbonate mixed with isopropanol. This solution was used to create the calibration curve depicted in FIG. 1B using the equation showed in FIG. 1B, the amount of ATRA in the polymer was calculated by dissolving HA−ATRA in the same media and reading the Amax at 343 nm. Each sample was measured in triplicate.

The amount of ATRA found in the sample is considered as 0.9 wt %
Degree of substitution determined by NMR (DS)=0.8%.

### Example 3. Synthesis, Purification and Isolation and Preparation of Microparticles Containing Retinoic Acid Attached to HA (HA−ATRA)

Hyaluronic acid (2.0 g, 5 mmol) characterized by an average molecular weight of 19,800 g/mol was dissolved in 40 mL of distilled water. To that solution, 20 mL of isopropanol (IPA) was added. After the solution was homogeneous, triethylamine (1.395 mL, 2.5 mmol) and DMAP (31.5 mg, 0.031 mmol) were consequently added to the mixture under stirring. In a second reaction flask, 0.045 mg of retinoic acid (0.2 mmol, 0.03 eq to HA dimer) were dissolved in isopropanol (5 ml) and activated by 0.004 ml of benzoyl chloride (0.2 mmol, 0.03 eq to HA dimer) in the presence of 1.395 mL of triethylamine (TEA). The activation was carried out for 60 minutes at 5° C. in darkness, after that time the activated mixture was added to solution containing HA. The resulting solution was maintained at 5° C. for 3 h in darkness. A saturated solution of sodium chloride was added to the reaction to precipitate the polymer. After that, the polymer was washed with an excess of anhydrous IPA (50 mL). The product was washed four times with solutions of isopropanol: water 85% (v/v) (4×50 mL). Finally, the precipitate was washed two more times with isopropanol. The product was filtrated and solubilized in water in a final concentration of 0.5% (w/v). Finally, the product was spray-dried using a mini spray dryer Büchi Mini Spray Drier B-290, which operates in a co-current mode and is equipped with a 0.7 mm diameter two-fluid nozzle. (inlet temperature: 190° C.; outlet temperature 90° C., solution feed rate: 10 mL/min, atomization air flow rate of 0.5 kg/h in a spray chamber size 165 mm/600 mm. Powder samples were stored in closed sachets at room temperature immediately after spray-drying to limit moisture uptake of the samples between production and testing. The particle size distribution of the solid was measured by Scanning Electronic Microscopy (SEM). (Average size of the batch=1.5 (±) 0.5. μm)

Additionally, the concentration of ATRA in the polymer was determined by UV-Vis. For that experiments, retinoic acid used for the chemical modification was dissolved in basic media, consisting of sodium hydroxide, sodium hydrogen carbonate or sodium bicarbonate mixed with isopropanol. This solution was used to create the calibration curve depicted in FIG. 1B using the equation showed in FIG. 1B, the amount of ATRA in the polymer was calculated by dissolving HA−ATRA in the same media and reading the Amax at 343 nm. Each sample was measured in triplicate.

The amount of ATRA found in the sample is considered as 2.5 wt %
Degree of substitution determined by NMR (DS)=2.8%.

### Example 4. Synthesis, Purification and Isolation and Preparation of Microparticles Containing Retinoic Acid Attached to HA (HA−ATRA)

Hyaluronic acid (2.0 g, 5 mmol) characterized by an average molecular weight of 97,000 g/mol was dissolved in 40 mL of distilled water. To that solution, 20 mL of isopropanol (IPA) was added. After the solution was homogeneous, triethylamine (1.395 mL, 2.5 mmol) and DMAP (31.5 mg, 0.031 mmol) were consequently added to the mixture under stirring. In a second reaction flask, 0.045 mg of retinoic acid (0.2 mmol, 0.03 eq to HA dimer) were dissolved in isopropanol (5 ml) and activated by 0.004 ml of benzoyl chloride (0.2 mmol, 0.03 eq to HA dimer) in the presence of 1.395 mL of triethylamine (TEA). The activation was carried out for 60 minutes at 5° C. in darkness, after that time the activated mixture was added to solution containing HA. The resulting solution was maintained at 5° C. for 3 h in darkness. A saturated solution of sodium chloride was added to the reaction to precipitate the polymer. After that, the polymer was washed with an excess of anhydrous IPA (50 mL). The product was washed four times with solutions of isopropanol: water 85% (v/v) (4×50 mL). Finally, the precipitate was washed two more times with isopropanol. The product was filtrated and solubilized in water in a final concentration of 0.5% (w/v). Finally, the product was spray-dried using a mini spray dryer Büchi Mini Spray Drier B-290, which operates in a co-current mode and is equipped with a 0.7 mm diameter two-fluid nozzle. (inlet temperature: 190° C.; outlet temperature 90° C., solution feed rate: 10 mL/min, atomization air flow rate of 0.5 kg/h in a spray chamber size 165 mm/600 mm. Powder samples were stored in closed sachets at room temperature immediately after spray-drying to limit moisture uptake of the samples between production and testing. The particle size distribution of the solid was measured by Scanning Electronic Microscopy (SEM). Average size of the batch=1.55 (±) 0.5 μm. After that the microparticles were rehydrated in water to confirm the structure by NMR

Additionally, the concentration of ATRA in the polymer was determined by UV-Vis. For that experiments, retinoic acid used for the chemical modification was dissolved in basic media, consisting of sodium hydroxide, sodium hydrogen carbonate or sodium bicarbonate mixed with isopropanol. This solution was used to create the calibration curve depicted in FIG. 1B. using the equation showed in FIG. 1B, the amount of retinoic acid in the polymer was calculated by dissolving HA−ATRA in the same media and reading the Amax at 343 nm.

The amount of ATRA found in the sample is considered as 0.49% wt.
Degree of substitution determined by NMR (DS)=0.39%.

### Example 5. Synthesis, Purification and Isolation and Preparation of Microparticles Containing Retinoic Acid Attached to HA (HA−ATRA)

Hyaluronic acid (2 g, 5.0 mmol) characterized by an average molecular weight of 15,000 g/mol was dissolved in 40 mL of distilled water. To that solution, 20 mL of isopropanol (IPA) was added. After the solution was homogeneous, triethylamine (1.4 mL, 10 mmol) and DMAP (0.031 g, 0.25 mmol) were consequently added to the mixture under stirring. In a second reaction flask, retinoic acid (0.083 g, 0.3 mmol or 0.055 eq.) was dissolved in isopropanol (20 ml) and activated by 0.032 ml of benzoyl chloride (0.3 mmol or 0.055 eq.) in the presence of 1.4 mL (10 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (200 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). (Average size of the batch=1.4 (±) 0.5. μm).

The amount of retinoic acid in the polymer was calculated by dissolving HA−ATRA in basic aqueous solution by reading the Amax at 343 nm. Each sample was measured in triplicate.
The amount of ATRA found in the sample is considered as 1.2% wt.
Degree of substitution was determined as (DS)=1.0%.

### Example 6. Synthesis, Purification and Isolation and Preparation of Microparticles Containing Retinoic Acid Attached to HA (HA−ATRA)

Hyaluronic acid (10 g, 25.0 mmol) characterized by an average molecular weight of 17,000 g/mol was dissolved in 200 mL of distilled water. To that solution, 100 mL of isopropanol (IPA) was added. After the solution was homogeneous, triethylamine (10.4 mL, 75 mmol) and DMAP (0.153 g, 1.25 mmol) were consequently added to the mixture under stirring. In a second reaction flask, retinoic acid (0.751 g, 2.5 mmol corresponding to 0.10 eq to HA dimer) was dissolved in isopropanol (20 ml) and activated by 0.29 mL of benzoyl chloride (2.5 mmol corresponding to 0.10 eq. to HA dimer) in the presence of 10.4 mL (75 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (200 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). (Average size of the batch=1.3 (±) 0.8. μm).

The amount of retinoic acid in the polymer was calculated by dissolving HA−ATRA in basic aqueous solution by reading the Amax at 343 nm.
The amount of ATRA found in the sample is considered as 2.16% wt.
Degree of substitution was determined by NMR (DS)=1.89%.

### Example 7. Synthesis, Purification and Isolation and Preparation of Microparticles Containing on Retinoic Acid Attached to HA (HA−ATRA)

Hyaluronic acid (2.0 g, 5.0 mmol) characterized by an average molecular weight of 15,000 g/mol was dissolved in 40 mL of distilled water. To that solution, 20 mL of isopropanol (IPA) was added. After the solution was homogeneous, triethylamine (1.39 mL, 10 mmol) and DMAP (0.031 g, 0.25 mmol) were consequently added to the mixture under stirring. In a second reaction flask, retinoic acid (0.225 g, 0.8 mmol, corresponding to 0.15 eq to HA dimer) was dissolved in isopropanol (20 ml) and activated by 0.022 mL of benzoyl chloride (0.02 mmol, corresponding to 0.15 eq to HA dimer) in the presence of 0.0348 mL (2.5 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (200 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). Each sample was measured in triplicate (Average size of the batch=1.3 (±) 0.6. μm).

The amount of retinoic acid in the polymer was calculated by dissolving HA−ATRA in basic aqueous solution by reading the Amax at 343 nm.
The amount of ATRA found in the sample is considered as 3.57% wt.
Degree of substitution was determined as (DS)=3.02%.

### Example 8. Synthesis, Purification and Isolation and Preparation of Microparticles Containing Retinoic Acid Attached to HA (HA−ATRA)

Hyaluronic acid (0.5 g, 1.3 mmol) characterized by an average molecular weight of 15,000 g/mol was dissolved in 40 mL of distilled water. To that solution, 20 mL of isopropanol (IPA) was added. After the solution was homogeneous, triethylamine (0.348 mL, 2.5 mmol) and DMAP (0.031 g, 0.25 mmol) were consequently added to the mixture under stirring. In a second reaction flask, retinoic acid (0.113 g, 0.8 mmol, corresponding to 0.30 eq to HA dimer) was dissolved in isopropanol (20 ml) and activated by 0.044 mL of benzoyl chloride (0.05 mmol, corresponding to 0.30 eq to HA dimer) in the presence of 0.348 mL (2.5 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (200 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). The amount of ATRA in the polymer was calculated by dissolving HA−ATRA in basic aqueous solution by reading the Amax at 343 nm. Each sample was measured in triplicate.

The amount of ATRA found in the sample is considered as 3.58% wt.
Degree of substitution was determined as (DS)=3.4%.

### Example 9. Synthesis, Purification and Isolation and Preparation of Microparticles Containing Retinoic Acid Attached to HA (HA−ATRA)

Hyaluronic acid (0.5 g, 1.3 mmol) characterized by an average molecular weight of 15,000 g/mol was dissolved in 40 mL of distilled water. To that solution, 20 mL of isopropanol (IPA) was added. After the solution was homogeneous, triethylamine (0.348 mL, 2.5 mmol) and DMAP (0.031 g, 0.25 mmol) were consequently added to the mixture under stirring. In a second reaction flask, retinoic acid (0.113 g, 0.8 mmol, corresponding to 0.30 eq to HA dimer) was dissolved in isopropanol (20 ml) and activated by 0.044 mL of benzoyl chloride (0.05 mmol, corresponding to 0.30 eq to HA dimer) in the presence of 0.348 mL (2.5 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (200 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). The amount of retinoic acid in the polymer was calculated by dissolving HA−ATRA in basic aqueous solution by reading the Amax at 343 nm. Each sample was measured in triplicate.

The amount of ATRA found in the sample is considered as 3.58% wt.
Degree of substitution was determined as (DS)=3.4%.

### Example 10. Synthesis, Purification and Isolation and Preparation of Microparticles Containing Retinoic Acid Attached to HA (HA−ATRA)

Hyaluronic acid (0.5 g, 1.3 mmol) characterized by an average molecular weight of 15,000 g/mol was dissolved in 40 mL of distilled water. To that solution, 20 mL of isopropanol (IPA) was added. After the solution was homogeneous, triethylamine (0.348 mL, 2.5 mmol) and DMAP (0.031 g, 0.25 mmol) were consequently added to the mixture under stirring. In a second reaction flask, retinoic acid (0.113 g, 0.8 mmol, corresponding to 0.30 eq to HA dimer) was dissolved in isopropanol (20 ml) and activated by 0.044 mL of benzoyl chloride (0.05 mmol, corresponding to 0.30 eq to HA dimer) in the presence of 0.348 mL (2.5 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (200 mL). The product was washed several times with solutions of isopropanol: water 85% (v/v) (4×200 mL).

Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). The amount of retinoic acid in the polymer was calculated by dissolving HA−ATRA in basic aqueous solution by reading the Amax at 343 nm. Each sample was measured in triplicate.

Degree of substitution was determined as (DS)=5.54%.

### Example 11. Synthesis, Purification and Isolation and Preparation of Microparticles Containing Retinoic Acid Attached to HA (HA−ATRA)

Hyaluronic acid (2 g, 5 mmol) characterized by an average molecular weight of 15,000 g/mol was dissolved in 40 mL of distilled water. To that solution, 20 mL of isopropanol (IPA) was added. After the solution was homogeneous, triethylamine (0.348 mL, 2.5 mmol) and DMAP (0.031 g, 0.25 mmol) were consequently added to the mixture under stirring. In a second reaction flask, retinoic acid (0.526 g, 0.8 mmol, corresponding to 0.035 eq to HA dimer) was dissolved in isopropanol (20 ml) and activated by 0.25 mL of benzoyl chloride (0.35 mmol, corresponding to 0.35 eq to HA dimer) in the presence of 0.348 mL (2.5 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (400 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). The amount of retinoic acid in the polymer was calculated by dissolving HA−ATRA in basic aqueous solution by reading the Amax at 343 nm. Each sample was measured in triplicate.

Degree of substitution was determined as (DS)=5.86%.

### Example 12. Synthesis, Purification and Isolation and Preparation of Microparticles Containing Retinoic Acid Attached to HA (HA−ATRA)

Hyaluronic acid (2 g, 5 mmol) characterized by an average molecular weight of 13,000 g/mol was dissolved in 40 mL of distilled water. To that solution, 20 mL of isopropanol (IPA) was added. After the solution was homogeneous, triethylamine (0.348 mL, 2.5 mmol) and DMAP (0.031 g, 0.25 mmol) were consequently added to the mixture under stirring. In a second reaction flask, retinoic acid (0.526 g, 0.8 mmol, corresponding to 0.035 eq to HA dimer) was dissolved in isopropanol (20 ml) and activated by 0.25 mL of benzoyl chloride (0.35 mmol, corresponding to 0.35 eq to HA dimer) in the presence of 0.348 mL (2.5 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (400 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). The amount of retinoic acid in the polymer was calculated by dissolving HA−ATRA in basic aqueous solution by reading the Amax at 343 nm. Each sample was measured in triplicate.

Degree of substitution was determined as (DS)=6.41%.

### Example 13. Synthesis, Purification and Isolation and Preparation of Microparticles Containing on Retinoic Acid Attached to HA (HA−ATRA)

Hyaluronic acid (0.5 g, 1.3 mmol) characterized by an average molecular weight of 97,000 g/mol was dissolved in 40 mL of distilled water. To that solution, 20 mL of isopropanol (IPA) was added. After the solution was homogeneous, triethylamine (0.348 mL, 2.5 mmol) and DMAP (0.031 g, 0.25 mmol) were consequently added to the mixture under stirring. In a second reaction flask, retinoic acid (0.113 g, 0.8 mmol, corresponding to 0.30 eq to HA dimer) was dissolved in isopropanol (20 ml) and activated by 0.044 mL of benzoyl chloride (0.05 mmol, corresponding to 0.30 eq to HA dimer) in the presence of 0.348 mL (2.5 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (200 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). Each sample was measured in triplicate. The amount of retinoic acid in the polymer was calculated by dissolving HA−ATRA in basic aqueous solution by reading the Amax at 343 nm.

The amount of ATRA found in the sample is considered as 4.1% wt.
Degree of substitution was determined by NMR as (DS)=4.0%.

### Example 14. Synthesis, Purification and Isolation and Preparation of Microparticles Containing Retinoic Acid Attached to HA (HA−ATRA)

Hyaluronic acid (0.5 g, 1.3 mmol) characterized by an average molecular weight of 97,000 g/mol was dissolved in 40 mL of distilled water. To that solution, 20 mL of isopropanol (IPA) was added. After the solution was homogeneous, triethylamine (0.523 mL, 2.5 mmol) and DMAP (0.008 g, 0.25 mmol) were consequently added to the mixture under stirring. In a second reaction flask, retinoic acid (0.113 g, 0.4 mmol, corresponding to 0.35 eq to HA dimer) was dissolved in isopropanol (20 ml) and activated by 0.044 mL of benzoyl chloride (0.4 mmol, corresponding to 0.35 eq to HA dimer) in the presence of 0.523 mL (2.5 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (200 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). Each sample was measured in triplicate. The amount of retinoic acid in the polymer was calculated by dissolving HA−ATRA in basic aqueous solution by reading the Amax at 343 nm.

The amount of ATRA found in the sample was determined as 6.9% wt.
Degree of substitution was determined by NMR (DS)=6.1%.

### Example 15. Synthesis, Purification and Isolation and Preparation of Microparticles Containing Retinoic Acid Attached to HA (HA−ATRA)

Hyaluronic acid (0.5 g, 1.3 mmol) characterized by an average molecular weight of 97,000 g/mol was dissolved in 40 mL of distilled water. To that solution, 20 mL of isopropanol (IPA) was added. After the solution was homogeneous, triethylamine (0.523 mL, 2.5 mmol) and DMAP (0.008 g, 0.25 mmol) were consequently added to the mixture under stirring. In a second reaction flask, retinoic acid (0.113 g, 0.4 mmol, corresponding to 0.40 eq to HA dimer) was dissolved in isopropanol (20 ml) and activated by 0.044 mL of benzoyl chloride (0.4 mmol, corresponding to 0.40 eq to HA dimer) in the presence of 0.523 mL (2.5 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (200 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). Each sample was measured in triplicate. The amount of retinoic acid in the polymer was calculated by dissolving HA−ATRA in basic aqueous solution by reading the Amax at 343 nm as 10 μg/mL.

The amount of ATRA found in the sample is considered as 4.9% wt.
Degree of substitution was determined by NMR (DS)=5.4%.

### Example 16. Synthesis, Purification and Isolation and Preparation of Microparticles Containing Retinoic Acid Attached to HA (HA−ATRA)

Hyaluronic acid (0.1 g, 0.3 mmol) of a mean molecular weight of 13,000 g/mol was dissolved in 2 mL of distilled water. To that solution, 1 mL of tetrahydrofuran (THF) was added. After the solution was homogeneous, triethylamine (0.10 mL, 0.8 mmol) and DMAP (0.002 g, 0.013 mmol) were consequently added to the mixture under stirring. In a second reaction flask, retinoic acid (0.075 g, 0.3 mmol) was dissolved in 2 ml of tetrahydrofuran (THF) and activated by benzoyl chloride (0.03 ml, 0.3 mmol) in the presence of 0.1 mL of triethylamine (TEA). The activation was carried out for 60 minutes at room temperature in darkness, after that time the activated mixture was added to solution containing HA. The resulting solution was stirred at room temperature (25° C.) for 8 h in darkness. A saturated solution of sodium chloride was added to the reaction to precipitate the polymer. After that, the polymer was washed with an excess of anhydrous IPA (10 mL). The product was washed several times with solutions of isopropanol: water 85% (v/v) (4×10 mL). Finally, the precipitate was washed two more times with isopropanol. The product filtrated by suction and solubilized in water in a final concentration of 0.5% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. The final solid concentration in the solvent mixture was fixed at 1 g/L. Powder samples were stored in closed sachets at room temperature immediately after spray-drying to limit moisture uptake of the samples between production and testing. The particle size distribution of the powders was measured by Scanning Electronic Microscopy (SEM). Each sample was measured in triplicate. The amount of retinoic acid in the polymer was calculated by dissolving HA−ATRA in basic aqueous solution by reading the Amax at 343 nm.

The amount of ATRA found was determined as 6.9% wt.
Degree of substitution determined by NMR is (DS)=7.1%.

### Example 17. Synthesis, Purification and Isolation and Preparation of Microparticles Containing Retinoic Acid Attached to HA (HA−ATRA)

Hyaluronic acid (0.1 g, 0.3 mmol) characterized by an average molecular weight of 97,000 g/mol was dissolved in 40 mL of distilled water. To that solution, 1 mL of isopropanol (IPA) was added. After the solution was homogeneous, triethylamine (0.105 mL, 0.8 mmol) and DMAP (0.002 g, 0.013 mmol) were consequently added to the mixture under stirring. In a second reaction flask, retinoic acid (0.038 g, 0.1 mmol, corresponding to 0.50 eq to HA dimer) was dissolved in isopropanol (1 ml) and activated by 0.015 mL of benzoyl chloride (0.1 mmol, corresponding to 0.50 eq to HA dimer) in the presence of 0.523 mL (2.5 mmol) of triethylamine (TEA). The activation was carried out for 60 minutes at 5° C. in darkness, after that time the activated mixture was added to solution containing HA. The resulting solution was maintained at 0° C. for 3 h in darkness. A saturated solution of sodium chloride was added to the reaction to precipitate the polymer. After that, the polymer was washed with an excess of anhydrous IPA (200 mL). The product was washed several times with solutions of isopropanol: water 85% (v/v) (4×200 mL). Finally, the precipitate was washed two more times with isopropanol. The product filtrated by suction and solubilized in water in a final concentration of 0.25% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). Each sample was measured in triplicate. The amount of retinoic acid in the polymer was calculated by dissolving HA−ATRA in basic aqueous solution by reading the Amax at 343 nm.

The amount of ATRA found in the sample is considered as 6.7% wt.
Degree of substitution was determined by NMR as (DS)=6.5%.

### Example 18. Synthesis, Purification and Isolation and Preparation of Microparticles Containing Retinoic Acid Attached to HA (HA−ATRA)

Hyaluronic acid of mean molecular weight of 97,000 g/mol (0.5 g, 1.3 mmol) was dissolved in 10 mL of distilled water. To that solution 10 mL of isopropanol (IPA) were added. After the solution was homogeneous, triethylamine (0.35 mL, 10 mmol) and DMAP (8 mg, 0.063 mmol) were consequently added to the mixture under stirring. In a second reaction flask, retinoic acid (0.113 g, 0.4 mmol) was dissolved in isopropanol (5 ml) and activated by 0.044 ml of benzoyl chloride in the presence of 0.35 of triethylamine (TEA). The activation was carried out for 60 minutes at room temperature in darkness, after that time the activated mixture was added to solution containing HA. The resulting solution was maintained at room temperature for 3 h in darkness. A saturated solution of sodium chloride was added to the reaction to precipitate the polymer. After that, the polymer was washed with an excess of anhydrous IPA (50 mL). The product was washed several times with solutions of isopropanol: water 85% (v/v) (4×50 mL). The product filtrated by suction and solubilized in water in a final concentration of 0.5% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing.

The degree of substitution (DS) was calculated by NMR and is defined as the number of retinoic acid molecules attached to 100 dimers of HA. The amount of retinoic acid in the polymer was calculated by dissolving HA−ATRA in basic aqueous solution by reading the Amax at 343 nm.

The amount of ATRA found in the sample is as 5.4% wt.
Degree of substitution (DS)=5.7%.

### Example 19. Synthesis, Purification and Isolation and Preparation of Microparticles Containing Retinoic Acid Attached to HA (HA−ATRA)

Hyaluronic acid characterized by a mean molecular weight of 270,000 g/mol (0.5 g, 1.3 mmol) was dissolved in 10 mL of distilled water. To that solution 10 mL of isopropanol (IPA) were added. After the solution was homogeneous, triethylamine (0.35 mL, 10 mmol) and DMAP (8 mg, 0.063 mmol) were consequently added to the mixture under stirring. In a second reaction flask, retinoic acid (0.056 g, 0.2 mmol) was dissolved in isopropanol (5 ml) and activated by 0.044 ml of benzoyl chloride in the presence of 0.35 of triethylamine (TEA). The activation was carried out for 60 minutes at room temperature in darkness, after that time the activated mixture was added to solution containing HA. The resulting solution was maintained at room temperature for 3 h in darkness. A saturated solution of sodium chloride was added to the reaction to precipitate the polymer. After that, the polymer was washed with an excess of anhydrous IPA (50 mL). The product was washed several times with solutions of isopropanol: water 85% (v/v) (4×50 mL). The product filtrated by suction and solubilized in water in a final concentration of 0.5% (w/v). The product was spray-dried using a mini spray dryer Büchi Mini Spray Drier B-290, which operates in a co-current mode and is equipped with a 0.7 mm diameter two-fluid nozzle. (inlet temperature: 180° C.; outlet temperature 100° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. Powder samples were stored in closed sachets at room temperature immediately after spray-drying to limit moisture uptake of the samples between production and testing. The particle size distribution of the powders was measured by Scanning Electronic Microscopy (SEM). Each sample was measured in triplicate. The amount of ATRA in the polymer was calculated by reading the λmax at 343 nm.

The amount of ATRA found in the sample is considered as 4.2% wt.
Degree of substitution determined by NMR (DS)=4.0%.

### Example 20. Synthesis, Purification and Isolation and Preparation of Microparticles Containing Retinoic Acid Attached to HA (HA−ATRA)

Hyaluronic acid of mean molecular weight of 470,000 g/mol (0.5 g, 1.3 mmol) was dissolved in 10 mL of distilled water. To that solution 10 mL of isopropanol (IPA) were added. After the solution was homogeneous, triethylamine (0.35 mL, 10 mmol) and DMAP (8 mg, 0.063 mmol) were consequently added to the mixture under stirring. In a second reaction flask, 0.1134 g of retinoic acid was dissolved in isopropanol (5 ml) and activated by 0.044 ml of benzoyl chloride in the presence of 0.35 of triethylamine (TEA). The activation was carried out for 60 minutes at room temperature in darkness, after that time the activated mixture was added to the solution containing HA. The resulting solution was maintained at room temperature for 3 h in darkness. A saturated solution of sodium chloride was added to the reaction to precipitate the polymer. After that, the polymer was washed with an excess of anhydrous IPA (50 mL). The product was washed several times with solutions of isopropanol: water 85% (v/v) (4×50 mL). The product filtrated by suction and solubilized in water in a final concentration of 0.5% (w/v). The product was spray-dried using a mini spray dryer Büchi Mini Spray Drier B-290, which operates in a co-current mode and is equipped with a 0.7 mm diameter two-fluid nozzle. (inlet temperature: 200° C.; outlet temperature 80° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. The final solid concentration in the solvent mixture was fixed at 1 g/L. Powder samples were stored in closed sachets at room temperature immediately after spray-drying to limit moisture uptake of the samples between production and testing. The amount of retinoic acid in the polymer was calculated by reading the λmax at 343 nm

The amount of ATRA found in the sample is considered as 0.54% wt.
Degree of substitution determined by NMR (DS)=0.5%

### Example 21. Synthesis, Purification and Isolation and Preparation of Microparticles Containing Retinoic Acid Attached to HA (HA−ATRA)

Hyaluronic acid of a mean molecular weight of 1,369,000 g/mol (0.5 g, 1.3 mmol) was dissolved in 10 mL of distilled water. To that solution 10 mL of isopropanol (IPA) were added. After the solution was homogeneous, triethylamine (0.35 mL, 10 mmol) and DMAP (8 mg, 0.063 mmol) were consequently added to the mixture under stirring. In a second reaction flask, 0.1134 g of retinoic acid was dissolved in isopropanol (5 ml) and activated by 0.044 ml of benzoyl chloride in the presence of 0.35 of triethylamine (TEA). The activation was carried out for 60 minutes at room temperature in darkness, after that time the activated mixture was added to solution containing HA. The resulting solution was maintained at room temperature for 3 h in darkness. A saturated solution of sodium chloride was added to the reaction to precipitate the polymer. After that, the polymer was washed with an excess of anhydrous IPA (50 mL). The product was washed several times with solutions of isopropanol: water 85% (v/v) (4×50 mL). The product was spray-dried using a mini spray dryer Büchi Mini Spray Drier B-290, which operates in a co-current mode and is equipped with a 0.7 mm diameter two-fluid nozzle. (inlet temperature: 200° C.; outlet temperature 85° C., solution feed rate: 10 mL/min, atomization air flow rate of 0.5 kg/h in a spray chamber size 165 mm/600 mm. HA−ATRA was were dissolved into water prior to spray-drying and the mixture maintained under moderate stirring while fed into the spray-dryer. The final solid concentration in the solvent mixture was fixed at 1 g/L. Powder samples were stored in closed sachets at room temperature immediately after spray-drying to limit moisture uptake of the samples between production and testing. The degree of substitution (DS) was calculated by NMR and is defined as the number of retinoic acid molecules attached to 100 dimers of HA. The integral of the anomeric proton HA signals from 4.4 to 4.8 was normalized to 67 and compared to the average of integral value of the signals located at δ=1.5, 1.63, 1.76 and 6.33 ppm, respectively corresponding to the unsaturations of retinoic acid and thus to the degree of substitution. The amount of retinoic acid in the polymer was calculated by dissolving HA−ATRA in basic aqueous solution by reading the Amax at 343 nm

The amount of ATRA found in the sample is considered as 2.12% wt.
Degree of substitution was determined by NMR as (DS)=1.8%.

### Example 22. Synthesis, Purification and Isolation and Preparation of HA Oligosaccharides and Retinoic Acid

Hyaluronic acid oligosaccharides (HA8^{NA}′ Mw 3,200 g/mol) (0.5 g, 1.3 mmol) were dissolved in 10 mL of distilled water. To that solution 10 mL of isopropanol (IPA) were added. After the solution was homogeneous, triethylamine (0.35 mL, 10 mmol) and DMAP (8 mg, 0.063 mmol) were consequently added to the mixture under stirring. In a second reaction flask, 0.1134 g of retinoic acid was dissolved in isopropanol (5 ml) and activated by 0.044 ml of benzoyl chloride in the presence of 0.35 of triethylamine (TEA). The activation was carried out for 60 minutes at room temperature in darkness, after that time the activated mixture was added to solution containing HA. The resulting solution was maintained at room temperature for 3 h in darkness. A saturated solution of sodium chloride was added to the reaction to precipitate the polymer. After that, the polymer was washed with an excess of anhydrous IPA (50 mL). The product was washed several times with solutions of isopropanol: water 85% (v/v) (4×50 mL). Finally, the precipitate was washed two more times with isopropanol. The product filtrated by suction and solubilized in water in a final concentration of 0.5% (w/v). The product was lyophilized. The degree of substitution (DS) was calculated by NMR and is defined as the number of retinoic acid molecules attached to 100 dimers of HA. The integral of the anomeric proton HA signals from 4.4 to 4.8 was normalized to 67 and compared to the average of integral value of the signals located at δ=1.5, 1.63, 1.76 and 6.33 ppm, respectively corresponding to the unsaturations of retinoic acid and thus to the degree of substitution. The product was separated by HPLC.

The amount of ATRA found in the sample was determined as 9.0% wt.

### Example 23. Stability Studies of Microparticles Made Thereof

Stability studies of HA−ATRA were performed after the process was completely optimized using five independent batches. The effect of degree of substitution was evaluated. Thus, a set of samples of microparticles prepared according to the method stated in the Example 1, (modification of the method as stated in Example 1 to get the different DS of conjugate is clear for a person skilled in the art), characterized by different degree of substitution DS around 0.5, 1.0, 2.0, 3.0 and 6.0% and Mw=15,000 g/mol of the ester derivative of the hyaluronan. This set of samples were packed in 5 g pouches with an inner lining of polyethylene film. Pouches were welded to become airtight and closed to avoid as much as possible the presence of air (A). Samples were submitted to 25±2° C. and 40% RH ±5% in validated climate chambers (Binder, Germany) according to ICH Q1A(R), guide of industry. A second set of samples was used for evaluation of storage temperature by incubation at −20±3.0 (for later storage of samples in freezer). The stability of the microparticles are resumed in Tables 1,2.

TABLE 1
Long-term stability of the microparticles determined at 25° C. (up to
12 months). The microparticles are characterised by an increased degree of
substitution which was obtained by using an increased molar amount of mixed
anhydride in the reaction (defined as eq. to HA dimer). M means a month.
Temperature (25° C.)
DS (determined by UV)

| Entry | Eq. | 0 M | 1 M | 2 M | 3 M | 4 M | 5 M | 6 M | 12 M |
|---|---|---|---|---|---|---|---|---|---|
| Exp. 1 | 0.03 | 0.54 | 0.45 | 0.48 | 0.43 | 0.45 | 0.45 | 0.47 | 0.44 |
| Exp. 2 | 0.03 | 0.58 | 0.56 | 0.50 | 0.53 | 0.56 | 0.55 | 0.51 | 0.50 |
| Exp. 3 | 0.03 | 0.39 | 0.33 | 0.33 | 0.33 | 0.34 | 0.37 | 0.38 | 0.35 |
| Exp. 4 | 0.06 | 1.08 | 1.08 | 1.02 | 1.04 | 1.11 | 1.08 | 1.08 | 1.05 |
| Exp. 5 | 0.06 | 1.07 | 1.12 | 1.10 | 1.06 | 1.02 | 1.07 | 1.12 | 1.07 |
| Exp. 6 | 0.10 | 1.89 | 2.04 | 1.99 | 2.20 | 1.99 | 1.89 | 1.88 | 1.89 |
| Exp. 7 | 0.10 | 2.05 | 1.90 | 1.86 | 1.88 | 1.83 | 2.05 | 1.90 | 1.89 |
| Exp. 8 | 0.15 | 2.87 | 2.87 | 2.82 | 2.92 | 2.85 | 2.87 | 2.87 | 2.85 |
| Exp. 9 | 0.15 | 3.04 | 2.52 | 2.60 | 2.64 | 2.53 | 3.04 | 2.52 | 2.52 |
| Exp. 10 | 0.30 | 4.41 | 2.17 | 1.65 | Change | Change | Change | Change | Change |
| | | | | | of λmax | of λmax | of λmax | of λmax | of λmax |
| Exp. 11 | 0.30 | 3.86 | 1.79 | 1.36 | Change | Change | Change | Change | Change |
| | | | | | of λmax | of λmax | of λmax | of λmax | of λmax |
| Exp. 12 | 0.30 | 4.80 | 2.54 | 1.58 | Change | Change | Change | Change | Change |
| | | | | | of λmax | of λmax | of λmax | of λmax | of λmax |
| Exp. 13 | 0.35 | 5.60 | 3.86 | 3.6 | Change | Change | Change | Change | Change |
| | | | | | of λmax | of λmax | of λmax | of λmax | of λmax |
| Exp. 14 | 0.40 | 6.20 | 4.8 | 5.0 | Change | Change | Change | Change | Change |
| | | | | | of λmax | of λmax | of λmax | of λmax | of λmax |
| Exp. 15 | 0.50 | 7.10 | 5.5 | 3.2 | Change | Change | Change | Change | Change |
| | | | | | of λmax | of λmax | of λmax | of λmax | of λmax |

TABLE 2
Long term stability of the microparticles was
also determined at −20° C. M means a month.
Temperature (−20° C.)
DS (determined by UV)

| Samples | 0 M | 1 M | 2 M | 3 M | 4 M | 5 M | 6 M | 12 M |
|---|---|---|---|---|---|---|---|---|
| Exp. 16 | 0.54 | 0.54 | 0.47 | 0.49 | 0.53 | 0.54 | 0.51 | 0.51 |
| Exp 17 | 0.58 | 0.58 | 0.57 | 0.53 | 0.59 | 0.60 | 0.58 | 0.61 |
| Exp 18 | 0.39 | 0.38 | 0.33 | 0.32 | 0.36 | 0.40 | 0.36 | 0.38 |
| Exp. 19 | 6.41 | 6.32 | 6.17 | 6.25 | 6.32 | 6.25 | 6.20 | 6.35 |
| Exp 20 | 5.86 | 5.75 | 5.72 | 5.74 | 5.55 | 5.69 | 5.75 | 5.80 |
| Exp 21 | 5.80 | 5.54 | 5.50 | 5.59 | 5.64 | 5.78 | 5.60 | 5.48 |

The stability of the conjugate HA−ATRA was demonstrated by thermal analyses and structural analyses were carried out by NMR. In brief, the TGA (Thermogravimetric analyses) was performed on the microparticles on a differential scanning calorimeter (DSC, Universal TA instruments). About 2 mg of powder was accurately weighed samples were loaded into aluminum pans and analyzed. The TGA runs were conducted from 20 to 600° C. at a speed of 10° C./min.

### Example 24. Gene Expression of Luciferase Reporter Under RARE Element

P19 cells stably expressing a luciferase reporter were maintained in a culture as previously described (Neuro Endocrinol Lett. 2008 October; 29(5):770-4. Alternation of retinoic acid induced neural differentiation of P19 embryonal carcinoma cells by reduction of reactive oxygen species intracellular production). The cells were treated with ATRA, HA−ATRA of varying degrees of substitution and ATRA mixed with HA. Concentrations of the compounds were also varied and corresponded to the molarity of retinoic acid present in each sample. The cells were treated for 6 hours and then assayed with Luciferase Reporter Gene Assay, high sensitivity (Sigma-Aldrich, St. Louis, Mo., USA) using EnVision plate reader (Perkin Elmer, Waltham, Mass., USA), the results are given in FIG. 9.

### Example 25. Expression of Genes Involved in Cholesterol Synthesis

This example illustrates the expressional changes in keratinocyte cholesterol metabolism pathway components (upon treatment with HA−ATRA (prepared as described in examples 5, 9), unbound ATRA and HA (HA+ATRA), hyaluronan (HA), untreated control (CTRL), retinoic isomers (13-cis-RET) and 9 cis (9-cis-RET). The HaCaT keratinocyte cells were individually treated with the compounds described below for 48 hours and sampled in the indicated times. The mRNA expression of HMGCS1, SQLE and DHRS3 was analyzed with quantitative real-time PCR (QRT-PCR) using a StepOnePlus (ThermoFisher, Waltham, Mass., USA). Briefly, 500 ng of total RNA was transcribed to cDNA (High-Capacity cDNA Reverse Transcription Kit, ThermoFisher, Waltham, Mass., USA). Approximately 5 ng of cDNA was used for QRT-PCR reaction in 10 μl volume. The TaqMan assays (all from ThermoFisher, Waltham, Mass., USA) used were: HMGCS1 (Hs00940429_m1), SQLE (Hs01123768_m1), DHRS3 (Hs01044021_m1) and RPL13A (Hs04194366_g1). Duplicate reaction tubes were set up for each sample. All expression values for HMGCS1, SQLE and DHRS3 were related to the amount of the housekeeping gene RPL13A to correct for variations in RNA levels and efficiency in cDNA synthesis. Regarding the analyzed enzyme involved in cholesterol synthesis, only treatment with microparticles of HA−ATRA increased the expression of HMGCS1 and SQLE, all samples containing retinoids increased expression of positive control DHRS3 (FIG. 10). However, microparticles HA−ATRA can upregulate cholesterol synthesis gene HMGCS1 like when the molarity of the retinoic acid bound to HA is the same in both treatments, this is shown in FIG. 11.

### Example 26. Cytotoxicity of the HA−ATRA Microparticles

The interaction of cells with modified HA derivatives is essential to be investigated before the product application. After chemical modification of HA, the derivatives should not be cytotoxic. In this work, the cytotoxicity was assessed using dilution method. The cell toxicity of prepared HA derivatives was tested at Normal Human Dermal Fibroblasts (NHDF) cells and NIH-3T3 cells. Cells were seeded into wells of 96-well test plates and cultured for 24 hours. Cell viability was measured 0, 24, 48, and 72 hours after treatment using the 3-(4,5-dimethylthiazol-2-yl)-2,5-diphenyl-tetrazolium bromide (MTT) assay. MTT stock solution (20 μL; of concentration 5 mg mL⁻¹) was added to cell culture medium (200 μL) in each well. The plates were incubated for 2.5 h at 37° C. Then, after removing of the MTT solution, 220 μL of lysis solution was added and lysis was carried out for 30 min at room temperature and the optical density was measured by Microplate reader VERSAmax at 570 nm. Derivatives of example 5 and 9 were assayed and found to be not cytotoxic up to concentration of 1,000 μgmL⁻¹. As an example, the results for HA−ATRA derivative are shown in FIG. 8, wherein negligible effects, and no significant differences in cell viability after 24, 48 or 72 h were observed in the whole concentration range tested, indicating an excellent cytocompatibility of the conjugate HA−ATRA.

### Example 27. Skin Penetration of HA−ATRA

Skin penetration experiments were performed according to OECD guidelines in vertical Franz diffusion cells using full-thickness skin (approx. 1 mm) from porcine auricles donated by local slaughter house. The receptor was filled with PBS (pH 7.4) held at 37° C., the excised tissue was clamped between donor and receptor with stratum corneum facing upward and exposing a diffusion area of 1 cm². After 30 min equilibration, the donor was slowly filled with 0.5 mL of control or HA−ATRA microparticles rehydrated with PBS and loaded with Nile red, in order to detect the fluorescence (c=1 mg/mL) or control solutions (containing 0.0010 or 0.0030 mg/mL Nile red) and covered by Parafilm. After an application lasting 5 and 20 h, the cells were dismantled, the skin was washed with PBS and (i) freezed and cryo-sectioned for further microscopic examination (FIG. 12).

### Example 28. Determination of Antioxidant Activity of HA−ATRA Described in Example 6

NIH 3T3 fibroblasts were seeded on 96-well panel and incubated with 100 μg/ml of microparticles (HA−ATRA) for 18 h. Furthermore, the cells were treated with dichlorofluorescein diacetate (DHA DA), which penetrates to cells and oxidizes to fluorescent dichlorofluorescein. After 30 min cells were treated either with 0.15 J/cm² and 0.3 J/cm² or 1 mM H₂O₂. Fluorescence intensity was measured after 30 min of treatment. Cells incubated with HA−ATRA generated less ROS in comparison with the reference, which were cells incubated in NHDF medium.

The second method used for evaluation of antioxidant activity was DPPH assay. 2,2-diphenyl-1-picrylhydrazyl (DPPH) is a dark-colored crystalline powder composed of stable free-radical molecules, that in presence of antioxidant change dark color to yellow. The results are measured colorimetrically.

### Example 29. Determination of Antioxidant Activity of HA−ATRA Described in Example 9

NIH 3T3 fibroblasts were seeded on 96-well panel and incubated with 100 μg/ml of microparticles (HA−ATRA) for 18 h. Furthermore, the cells were treated with dichlorofluorescein diacetate (DHA DA), which penetrates to cells and oxidizes to fluorescent dichlorofluorescein. After 30 min cells were treated either with 0.15 J/cm² and 0.3 J/cm² or 1 mM H₂O₂. Fluorescence intensity was measured after 30 min of treatment. Cells incubated with HA−ATRA generated less ROS in comparison with the reference, which were cells incubated in NHDF medium.

The second method used for evaluation of antioxidant activity was DPPH assay. 2,2-diphenyl-1-picrylhydrazyl (DPPH) is a dark-colored crystalline powder composed of stable free-radical molecules, that in presence of antioxidant change dark color to yellow. The results are measured colorimetrically.

### Example 30. Induction of Collagen

WS1 fibroblasts were incubated with different concentration of microparticles HA−ATRA for 22 h. The induction of collagen 1 expression was observed after qPCR analysis.

### Example 31. Induction of Elastin

WS1 fibroblasts were incubated with different concentrations of microparticles HA-ATRA for 22 h. The induction of elastin expression was observed after qPCR analysis.

### Example 32. Induction of Fibronectin

WS1 fibroblasts were incubated with different concentrations of microparticles HA−ATRA for 22 h. The induction of fibronectin was observed after immunofluorescence staining and visualized by confocal microscopy.

### Example 33. Antimicrobial Activity Assay

Streptococcus epidermidis and Bacillus subtilis were seeded on tryptic soy agar (TSA, recommended for use as a general growth medium for the isolation and cultivation of microorganisms), and on TSA supplemented with 1 (w/v) % of HA−ATRA. After 24 h of incubation there were no colonies of B. subtilis and less colonies of S. epidermidis grown on TSA enriched with 1% (w/v) of microparticles HA−ATRA.

### Example 34. Dermal Irritation Test In Vivo

The dermal irritation test was performed in occlusion on a forearm of 15 volunteers. The microparticles of HA−ATRA was dissolved in PBS at two concentrations (500 and 1000 μg/ml) and applied for 18 h. After application we did subjective evaluation (erythema, edema) of the results at different time points: 0, 2 h, 24 h, 48 h, 72 h. HA−ATRA did not show an irritating activity on skin. The data were evaluated according to the table (FIG. 21):

Primary dermal irritation index

| | |
|---|---|
| Non-irritating | PDII < 0.5 |
| Mildly irritating | PDII ≥ 0.5 |
| Moderately irritating | PDII ≥ 3.0 |
| Severely/Extremely irritating | PDII ≥ 5.0 |

### Example 35. Development of Nanoemulsion Made of HA−ATRA

Nanoemulsions were prepared using the method of homogenization under high agitation by Ultra-Turrax® equipment (IKA, Germany). The formulation consisted of an oil phase containing an essential oil and sorbitan monooleate (2%), and an aqueous phase containing microparticles of HA−ATRA (2% w/v) and ultrapure water. The phases were homogenized separately with the aid of a magnetic stirrer, then the oil phase was injected into the aqueous phase under agitation of 10,000 rpm, which was increased to 17,000 rpm and sustained for 30 min with temperature control.

### Example 36. Formulation of Hydrogel Containing HA−ATRA

A solution of oxidized HA (HA-OX) prepared according to the patent WO2011069475A2 and HA−ATRA microparticles (1:1) was prepared in demineralized water in which the final concentrations of the polymers were from 1.5 to 7.5% (w/v), respectively. To that solution was added (0.1% w/v) of O,O′-1,3-propanediylbishydroxylamine dihydrochloride 98% linker was dissolved and homogenised. Then, the solution was transferred to Teflon molds (cylinders, diameter 10 mm, height 5 mm).

### Example 37: Face Cream Formulation Prepared in Base of Microparticles Made of HA−ATRA

(a) from 0.001 to 0.1% by weight of active ingredient or HA−ATRA,
(i) at least one fat selected from the group consisting of natural, modified or synthetic fatty acids or its derivative,
(ii) at least one nonionic surfactant and emulsifier,
(iii) at least one oil or vegetable extract,
(iv) at least one alcohol, and
(v) at least one moisturizer;
(b) 6.0 to 32.0% by weight of cosmetically acceptable additives; and
(c) q.s.p. 100% by weight of hydrophilic gel-cream base or water.
Three examples of cosmetic formulations are resumed on Tables a, b and c (below).

TABLE a

| ingredient | % | INCI |
|---|---|---|
| Ercarel TCC V | 12 | Caprilyc/Capric triglyceride |
| Sorbitan Stearate | 1.5 | Sorbitan monostearate |
| Polysorbate 60 | 2.5 | Polysorbate-60 |
| Shea Butter | 4.5 | Butyrospermum Parkii Fruit |
| Cetyl Alcohol | 4 | Cetyl Alcohol |
| Stearic Acid | 2 | Stearic acid |
| Water deionized | 70.1 | Aqua |
| Glycerin | 2 | Glycerin |
| EDTA | 0.2 | Tetrasodium EDTA |
| HA-ATRA | 0.01 | |
| Benzylalkohol-DHA | 0.8 | Benzylalcohol, dehydroacetic acid |
| 20% TEOA | 0.4 | Triethanolamine |

TABLE b

| ingredient | % | INCI |
|---|---|---|
| Glycerin | 4 | Glycerin |
| Jojoba oil | 12 | Simmondsia chinensis seed oil |
| Cocoa butter | 6 | Theobroma cacao seed butter |
| Cream maker Blend | 3 | Glyceryl stearate, PEG-100 |
| | | stearate |
| Stearic acid | 2 | Stearic acid |
| Cetyl alcohol | 3 | Cetyl Alcohol |
| Vitamin E acetate | 1 | Tocopheryl Acetate |
| 20% Triethanolamine | 0.55 | Triethanolamine |
| (TEOA) | | |
| Water deionized | 67.64 | Aqua |
| HA-ATRA | 0.1 | |
| Benzyl alcohol DHA | 0.8 | Benzylalcohol, dehydroacetic acid |

TABLE c

| ingredient | % | INCI |
|---|---|---|
| Triglyceride | 12 | Caprilyc/Capric triglyceride |
| Avocado butter | 6 | Hydrogenated avocado oil |
| TEGO Care CG 90 | 4 | Cetearyl Polyglycoside |
| Stearic acid | 2 | Stearic Acid |
| vit E acetate | 1 | Tocopheryl Acetate |
| Water deionized | 71.19 | Aqua |
| Glycerin | 2 | Glycerin |
| HE-cellulose | 1 | Hydroxyethylcellulose |
| HA-ATRA | 0.05 | |
| Benzylalcohol-DHA | 0.8 | Benzylalcohol, dehydroacetic acid |

### Example 38. Encapsulation of Hydrophobic Compounds in HA−ATRA

Resveratrol (9 mg) was dissolved in 3 mL of methanol and mixed rehydrated microparticles made of HA−ATRA (1% wt). Solvents were removed under reduced pressure. Resulting film was rehydrated with water, filtered through a 0.1 μm glass fiber to remove unincorporated compound and freeze-dried.

The encapsulated amount was determined by UV-Vis after breakage of the nano delivery system. 1.44% wt. Resveratrol.

### Example 39. Encapsulation of Hydrophobic Compounds in HA−ATRA

Resveratrol (10 mg) was dissolved in 3 mL of ethanol and mixed with rehydrated microparticles made of HA−ATRA (1% wt). Solvents were removed under reduced pressure. Resulting film was rehydrated with water, filtered through a 0.1 μm glass fiber to remove unincorporated compound and freeze-dried.

The encapsulated amount was determined by UV-Vis after breakage of the nano delivery system. 2.5% wt. Resveratrol.

### Example 40. Encapsulation of Hydrophobic Compounds in HA−ATRA

Curcumin (5-12.5 mg) was dissolved in 3 mL of ethanol and mixed with rehydrated microparticles made of HA−ATRA (1% wt). Solvents were removed under reduced pressure. Resulting film was rehydrated with water, filtered through a 0.1 μm glass fiber to remove unincorporated compound and freeze-dried.

The encapsulated amount was determined by UV-Vis after breakage of the nano delivery system.

0.5% wt. curcumin

### Example 41. Encapsulation of Hydrophobic Compounds in HA−ATRA

Retinyl palmitate (10 mg) was dissolved in 3 mL of isopropanol and mixed with rehydrated particles made of HA−ATRA (1% wt). Solvents were removed under reduced pressure. Resulting film was rehydrated with water, filtered through a 0.1 μm glass fiber to remove unincorporated compound and freeze-dried.

The encapsulated amount was determined by HPLC after breakage of the nano delivery system. 7.6% wt. retinyl palmitate.

1. A composition comprising microparticles based on ester derivatives of hyaluronan, the microparticles comprising a conjugate of all-trans retinoic acid and hyaluronan of the general formula I: 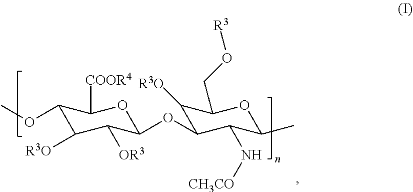
wherein n is integer in the range of from 1 to 5000 dimers,
each R⁴ is H⁺ or a pharmaceutically acceptable salt,
each R³ is —H or an all-trans retinoic acid residue of the formula II, where [symbol] is in the place of covalent bond of all-trans retinoic acid residue of the formula II 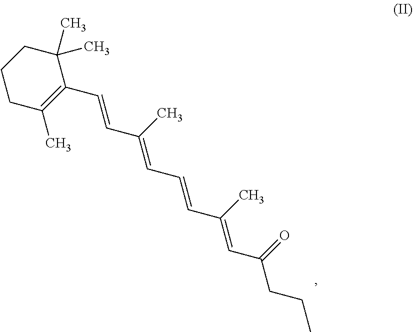 [SMILES: CCCC(=O)/C=C(C)/C=C/C=C(C)/C=C/C1=C(C)CCCC1(C)C]
with the proviso that at least one R³ of the conjugate is the all-trans retinoic acid residue of the formula II, and wherein the degree of substitution of the all-trans retinoic acid residues of the formula II in the conjugate of hyaluronan is in the range of from 0.1 to 8%.

2. The composition of claim 1, wherein in the microparticles the conjugate of the formula I comprises a molar weight in the range of from 3,200 to 100,000 g/mol.

3. The composition of claim 1, wherein in the microparticles the conjugate of the formula I comprises a degree of substitution of the all-trans retinoic acid residues of the formula II in the range from 0.5 to 8%, and a weight in the range of from 6,000 to 30,000 g/mol.

4. The composition of claim 1, wherein in the microparticles the conjugate of the formula I comprises a degree of substitution in the range of from 0.3 to 3.1%, and a molar weight in the range of from 6,000 g/mol to 20,000 g/mol.

5. The composition of claim 1, wherein in the microparticles at least one R⁴ comprises a pharmaceutically acceptable salt selected from the group of ions of alkali metals and ions of alkaline-earth metals.

6. The composition of claim 1, wherein the microparticles comprise an average diameter in the range of from 500 nm to 5 μm.

7. A method of preparing the composition of claim 1, said method comprising:
reacting an activated all-trans retinoic acid with a hyaluronic acid or a pharmaceutically acceptable salt thereof in the presence of an organic base,
wherein the activated all-trans retinoic acid is of the general formula III 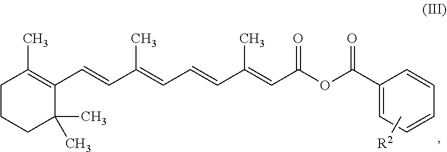
where R² represents one or more substituents selected from the group of H, —NO₂, —COOH, halides, and C₁-C₆ alkylkoxy groups; and
wherein the reaction is carried out in a mixture of water and water-miscible polar solvent in a ratio of from 99% to 50% v/v of water-miscible polar solvent, to form a solution comprising the conjugate of -all-trans retinoic acid and hyaluronan of the general formula I; and
spray-drying the solution using at inlet temperature of from 150 to 200° C. and an outlet temperature of from 80 to 100° C., thereby forming a composition comprising the microparticles of the conjugate of all-trans retinoic acid and hyaluronan of the general formula I.

8. The method of claim 7, wherein the concentration of the conjugate of -all-trans retinoic acid and hyaluronan in the solution is in the range of from 0.25 to 2.5% (w/v).

9. The method of claim 7, wherein the reaction of the activated all-trans retinoic acid of the formula III and the hyaluronic acid or the pharmaceutically acceptable salt thereof is carried out at a temperatures in the range of from 0 to 37° C., for a time of from 1 to 4 hours, in darkness.

10. The method of claim 7, wherein the organic base comprises an aliphatic amine having a linear or branched, saturated or unsaturated, C₃-C₃₀ alkyl group; and wherein the polar solvent is selected from the group of isopropanol, dimethyl sulfoxide, tert-butanol, dioxane, and tetrahydrofuran.

11. The method of claim 10, wherein the organic base is N,N-diisopropylethylamine, triethylamine, or dimethylaminopyridine, and wherein the polar solvent is isopropanol.

12. The method of claim 7, wherein 0.01 to 2.0 molar equivalents of the activated all-trans retinoic acid of the formula III is reacted with 1 molar equivalent of a dimer of hyaluronic acid.

13. The method of claim 7, further comprising preparing the activated all-trans retinoic acid of the formula III by reaction of all-trans retinoic acid with an activation agent in the presence of an organic base and a mixture of water and a water-miscible polar solvent, wherein the activation agent comprises a substituted or non-substituted benzoyl chloride or derivative thereof having the general formula IV 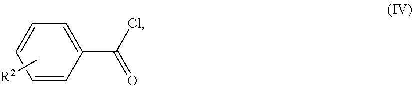
wherein R² represents one or more substituents selected from H, —NO₂, —COOH, halides, and C₁-C₆ alkoxy groups.

14. The method according to claim 13, wherein the all-trans retinoic acid is reacted with the activation agent at a temperature in the range of from 5 to 37° C., for a time of from 0.5 to 24 hours, in darkness.

15. The method of claim 13, wherein from 0.03 to 0.3 molar equivalents of the activation agent is used in the activation of the all-trans retinoic acid with respect to 1 molar equivalent of a hyaluronan dimer reacted with the activated all-trans retinoic acid formed thereby.

16. The method of claim 13, wherein: (i) the solvent is selected from the group of isopropanol, tert-butanol, dioxane, and tetrahydrofuran; (ii) the activation agent is benzoyl chloride; (iii) the organic base is selected from the group of N, N-diisopropylethylamine, triethylamine, trimethylamine, and dimethylaminopyridine; or (iv) any of (i)-(iii).

17. (canceled)

18. The composition of claim 1, comprising the conjugate of all-trans retinoic acid and hyaluronan of the general formula I in an amount in the range of from 0.001 to 20 wt. %, based on the total weight of the composition.

19. The composition of claim 18, further comprising at least one hydrophilic polymer in amount of from 1 to 75 wt. % based on the total weight of the composition.

20. The composition of claim 1, wherein the microparticles further comprise at least one hydrophobic compound encapsulated by the conjugate of all-trans retinoic acid and hyaluronan.

21. The composition of claim 1, further defined as: (i) a cosmetic or medicinal composition for improving epidermal barrier maintenance in skin that transcriptionally regulates lipid synthesis; (ii) an anti-aging composition for inducing collagen 1, fibronectin, and/or elastin expression; (iii) an antimicrobial composition effective against Gram-positive bacteria; or (iv) any of (i)-(iii).

22. (canceled)

23. (canceled)
